(19) United States
(12) Patent Application Publication
(10) Pub. No.: US 2003/0082571 A1
(43) Pub. Date: May 1, 2003

(54) Linear nucleic acid and sequence therefor

(76) Inventors: Edward Hanna Kachab, Queensland (AU); Graeme Ross Barnett, Queensland (AU)

Correspondence Address: HELLER EHRMAN WHITE & MCAULIFFE LLP, 1666 K STREET,NW, SUITE 300, WASHINGTON, DC, 20006, US

(21) Appl. No.: 10/117,108

(22) Filed: Apr. 8, 2002

Related U.S. Application Data
(60) Provisional application No. 60/282,491, filed on Apr. 10, 2001.

(51) Int. Cl. C12Q 1/68; C07H 21/04

(52) U.S. Cl. 435/6; 536/24.3

(57) ABSTRACT

Nucleic acids and sequences therefor are disclosed that are characterized by a reduction or lack of internal secondary structure, are capable of hybridizing with a complementary nucleic acid and do not hybridize with non-complementary nucleic acids (eg. do not cross-hybridize or form dimers) under low stringency hybridization conditions. In particular, the nucleotide sequences enable use of these nucleic acids, without reduction in target hybridization efficiency with increasing nucleic acid length. The nucleic acids may be used with analyte capture systems, for example medical, veterinary and agricultural diagnostic applications. In particular, the nucleic acid may be used as irrelevant binding pairs in an analyte capture system, such as an array or lateral flow assay.

### FIELD OF THE INVENTION

The present invention relates to linear nucleic acids, for example oligonucleotides, having nucleotide sequences enabling use of these nucleic acids, without reduction in target hybridisation efficiency with increasing nucleic acid length. The nucleic acids may be used for the discriminatory and concurrent cross-linking by hybridisation or hybridisation capture, of a multiplicity of species in solution, at a mixed solution-solid phase interface, at a solid-solid phase interface, or combination thereof under non-stringent or relaxed hybridisation conditions. The present invention is widely useful for diagnostic biomedical applications in the human, veterinary, agricultural and food sciences, as well as applications for high throughput screening, among others.

### BACKGROUND OF THE INVENTION

One of the most widely used capture systems in biomedicine and biotechnology is the biotin-avidin or streptavidin capture system (Wilchek, M. and Bayer, E. A., 1990, Methods Enzymol., 184 5; Wilchek, M. and Bayer, E. A., 1990, Methods Enzymol., 184 123). This is based on a highly specific and strong interaction between biotin, a water-soluble vitamin B₆, and avidin, a protein isolated from egg white. This system has found broad applications in areas such as cytochemistry (Bayer, E. A. et al., 1976, FEBS Lett., 68 240), gene mapping (Broker, T. R. et al., 1978, Nucleic Acids Res., 5 363), immunoassays (Guesdon, J. L. et al., 1979, J. Histochem. Cytochem., 27 1131; U.S. Pat. No. 5,126,241), immunotherapy (Philpott, G. W. et al., 1980, J. Immunol. 125, 1201), and lateral flow assays, for example, International Patent Publication No. WO 97/06439 and U.S. Pat. No. 6,037,127.

Another capture system which has found broad application in biology is based on metal chelate co-ordination chemistry. In this system a metal chelate with suitable co-ordination sites such as Ni²⁺ iminodiacetic acetic which forms a stable complex with a suitable donor ligand such as a hexahistidine tag (E. Sulkowski, 1989, BioEssays, 10 170; Petty, K. J., 1996, Metal-Chelate Affinity Chromatography. In: Current protocols in molecular biology, F. M. Ausubel (ed.), vol 2, John Wiley and Sons, New York), herein incorporated by reference. This system has found the greatest application in the technique of immobilised metal affinity chromatography (IMAC) for the purification of recombinant proteins (e.g. Porath, J., 1992, Prot. Expr. Purif., 3 263; Alnemri, et al., 1993, Proc. Natl. Acad. Sci., 90, 6839), herein incorporated by reference. It has also been applied for the capture and analysis of recombinant proteins with a His Tag directly on microtiter plates and magnetic beads (QIAexpress Assay System, Qiagen, GMBH, Germany). Recently, the capture of dendrimers conjugated to horseradish peroxidase and recombinant proteins with a His Tag has been demonstrated using this capture system on glass fibre filters derivatised with a metal chelate forming group, iminodiacetic acid (International Patent Publication No. WO/70012). Dendrimers conjugated to anti-analyte antibodies have also been used in immunoassays whereby the immunogenic complex has been captured on glass microfibre filters through the interaction between the dendrimer and the microfibre glass surface. In this case the capture system is based on charge interaction between the positive charges on the dendrimer and the negative charges on the glass filter as for U.S. Pat. No. 5,861,319 and International Patent Publication No. WO 99/32884.

In the examples described above these capture systems have been applied in assay platforms whereby one component of the capture system is usually conjugated to a ligand capable of binding specifically to the analyte (anti-analyte) with another component of the capture system being immobilised on a solid support. Thus, the capture system may comprise a single stage system having one binding pair or a multi-stage system having two or more binding pairs. Thus, the binding pair involving immobilisation to the solid support has a binding specificity which is irrelevant to or independent of the binding specificity of the analyte/anti-analyte interaction of interest. Examples of such applications can be found in International Patent Publication No. WO 97/06439, U.S. Pat. Nos. 5,126,241, 4,496,654, and 5,001,049.

Irrelevant binding pairs are usually used in various formats to supplement anti-analyte/analyte interactions, as shown, for example, to improve the efficiency of lateral flow assays as described in International Patent Publication No. WO 97/06439. In this reference streptavidin is bound to a capture zone in a test filter strip and an anti-analyte is attached to biotin. The analyte forms a complex sandwiched between the anti-analyte attached to biotin and label in solution. This complex is then captured on the test filter strip through biotin-streptavidin interaction. A strong affinity constant between biotin and streptavidin (Kₐ=10¹⁵ M⁻¹) allows the complex to be captured at a higher capture efficiently than having the anti-analyte directly immobilised onto the capture zone. This is regularly practised in many different lateral flow formats for example; European Patent No. EP0291194, U.S. Pat. Nos. 5,798,273, 5,607,863, 5,648,274, and 5,468,648.

Nucleic acids have been developed that bind with high affinity and selectivity to the disaccharide cellobiose (Engelke, D. R., et al., 1998, Proc. Natl. Acad. Sci., 95, 5462). These nucleic acids comprise between 36-41 base pairs and bind to cellulose comprising repeating units of cellobiose. This single stage capture system can potentially be used as an irrelevant binding pair in similar applications to the capture systems mentioned above. Other examples of single stage capture systems which could also potentially be employed as irrelevant binding pairs include, but not limited to, systems such as lectins, including concanavalin A, pea lectin, tomato lectin, etc., and their respective glycans (Osawa, T. and Tsuji, T., 1987, Annu. Rev. Biochem., 56, 21; Cummings, R. D., 1994, In Methods in enzymology, Vol. 230, (ed. W. J. Lennarz and G. W. Hart), p. 66. Academic Press, New York; Osawa, T., et al., 1995, Mol. Biotechnol., 3, 25), immunoglobulins and protein A or G (Gerston, D. M. and Marchalonis, J. J., 1978, J. Immunol. Methods, 24, 305), antigens and their respective antibodies, a hapten and an anti-hapten such as an immunoglobulin (Tijseen, P., 1985, Laboratory techniques in Biochemistry and Molecular Biology: Practice and Theory of Enzyme Immunoassays (ed. R. H. Burdon, and P. H. Knippenberg) Elsevier Science Publishers, Amsterdam), an antibody and a peptide mimetic (Still, W. C., 1996, Acc. Chem. Res., 29, 155), or folic acid and folate binding proteins. In theory, any two entities, which are either biologically or chemically derived, with an affinity for each other could potentially be employed as irrelevant binding pairs for a multitude of applications and in a multitude of different formats as generally known and practised in the art. A list of species, including binding pairs capable of forming covalent bonds, which may participate in such interactions are mentioned in International Patent Publication No. WO 99/32885, and U.S. Pat. No. 6,037,127 which are herein incorporated by reference.

Many different single stage capture systems are available for use as irrelevant binding pairs either for the capture of analytes or analyte/anti-analyte complex species or simply for the immobilisation of anti-analytes or other capture species on a solid support or in a sandwich format of some kind to facilitate detection of the analyte or analyte/anti-analyte complex (Eggers, M., et al., 1999, BioTechniques, 27, 778). It will be appreciated that these capture systems are limited in their applicability as irrelevant binding pairs to systems where multiple analytes, or multi-analyte/anti-analyte complexes or multiple capture species need to be either captured or immobilised on a solid support discretely at specific identifiable capture zones for each of the analytes or analyte/anti-analyte complexes or capture species from a single mixture in solution. In this respect, more than one capture system needs to be used concurrently to achieve the discrete capture needed. This is exemplified in the use of the biotin/streptavidin capture system in combination with metal chelation capture and/or capture based on charge interaction such as dendrimers and glassfibre filters as described above. A disadvantage of using more than one capture system is that different conditions are needed for optimal capture and discrimination for each capture system. Also, various modifications and derivatisation may be required depending on the capture system and capture support medium used, which makes the process impracticable and complicated.

Complementary nucleic acid binding pairs represent an additional capture system which may be employed as irrelevant binding pairs. This is based on hybridisation of two nucleic acids having complementary sequences, either in solution or in a mixed phase system, to form a stable duplex, with high specificity from Watson-Crick base pairing (Wetmur, J. G., 1991, Critical Reviews in Biochemistry and Molecular Biology, 26, 227). Since only nucleic acids having complementary sequences will hybridise with highest specificity and affinity, and since millions of different binding pairs of nucleic acids having sequences and different lengths can be generated combinatorially from the four basic building blocks of DNA, this capture system lends itself well for use in the applications described above, provided that nucleic acid pairs having complementary sequences do not cross-hybridise with other nucleic acid pairs having different complementary sequences under non-stringent or relaxed hybridisation conditions. Applications also include multiple analyte or multiple analyte/anti-analyte capture and detection and multiple analyte or capture species (for example antibody) immobilisation on various supports. Also, two or more species may be cross-linked by hybridisation of nucleic acids attached to each respective species. The vast advancement in nucleic acid chemistry over the past 15 years, includes automated synthesis of custom-made nucleic acids with many different reactive groups attached at either their 5′ or 3′ end, incorporating linkers of variable lengths, for a broad range of conjugation applications (Glen Research, Sterling, Va.) also makes this capture system a practical tool for the above applications.

One example in which oligonucleotides having complementary sequences are used as irrelevant binding pairs can be found in International Patent Publication No. WO 95/24649 incorporated herein by reference. In this reference two different oligonucleotides are conjugated to two different antibodies, each with a specificity to a different analyte. When these conjugates are mixed with analytes in solution, immunocomplexes form which are then captured through hybridisation of the tail oligonucleotide moiety attached to each antibody with its complementary oligonucleotide immobilised on a solid support allowing for selective analyte capture at specific microspots.

In International Patent Publication No. WO 95/24649, referred to above, two oligonucleotide pairs are disclosed. A first pair comprises an oligonucleotide having a poly-CA sequence and an oligonucleotide having a complementary sequence thereto. A second oligonucleotide pair comprises an oligonucleotide having a poly-GA sequence and an oligonucleotide having a complementary sequence thereto. These oligonucleotides appear to have been successfully used for discrete capture of two different analytes in solution on microspots. This reference makes no mention of how the nucleotide sequences of the oligonucleotides were derived, and makes no mention of other sequences to be used when a sample comprises more than two analytes. In addition, a preferred length of these oligonucleotides is disclosed as between 8 and 30 bases with use of very long nucleic acids not being preferred due to reduction in hybridisation specificity and formation of secondary structure.

Reference may also be made to International Patent Publication No. WO 99/32885, incorporated herein by reference, which discloses use of oligonucleotides as irrelevant binding pairs for capture of a multiplicity of analyte/anti-analyte complexes in a lateral flow assay format. This reference makes no mention of any specific nucleotide sequence which may be used in the practice of the invention.

Further reference may be made to U.S. Pat. No. 6,037,127, incorporated herein by reference. This reference provides an example for the capture and detection of three analytes in a lateral flow assay system by using oligonucleotide capture probes specific to each target immobilised on specific capture zones on a membrane. However, use of oligonucleotides as irrelevant binding pairs, particularly for the purposes of providing for a generic lateral flow strip for capture of any target nucleic acid is not contemplated in this invention.

International Patent Publication No. WO 00/58516 relates to a universal array comprising: i) a solid support, ii) an oligonucleotide tag, and iii) a locus-specific tagged oligonucleotide. The oligonucleotide tag is attached to the solid support and comprises a unique known arbitrary nucleotide sequence sufficient in length to hybridise to the locus-specific tagged oligonucleotide. The oligonuclotide tag is characterised by comprising a nucleotide sequence different from other oligonucleotide tags in the array and preferably does not interact with an amplified target nucleic acid. The locus-specific tagged oligonucleotide is characterised by comprising a 5′ end complementary to a unique arbitrary sequence of the oligonucleotide tag and a 3′ end capable of hybridising to a target nucleic acid and when hybrised thereto terminates one nucleotide 5′ to the target nucleic acid. There is no disclosure of how to pre-select a nucleotide sequence of either the oligonucleotide tag or locus-specific tagged oligonucleotide. In particular, there is no disclosure of predicting a desired nucleotide sequence for an oligonucleotide tag or locus-specific tagged oligonucleotide.

Efficiency of hybridisation between oligonucleotides having complementary nucleotide sequences increases with an increase in oligonucleotide length. For example, an oligonucleotide in solution and an oligonucleotide immobilised on a solid support matrix, such as a microchip, results in an increase in the observed signal with increasing nucleic acid length (Mirzabekov, A., et al., 1996, Nucleic Acids Research, 24, 2998; Mirzabekov, A., et al., 1997, Analytical Chemistry, 270, 203; herein incorporated by reference). However, the possibility of secondary structure and the formation of stable hairpin loops increases with increasing oligonucleotide length which decreases the efficiency of hybridisation making it necessary to use oligonucleotide modules to open up the secondary structure and increase the efficiency of hybridisation (Lundeberg, J., et al., 1998, Analytical Chemistry, 255, 195).

### SUMMARY OF THE INVENTION

There is a need for a nucleic acid comprising a nucleotide sequence which can be of any length, lack internal secondary structure, is capable of hybridising with a complementary nucleic acid and does not hybridise with a non-complementary nucleic acid (eg cross-hybridise or form dimers) at least under low stringency conditions. Preferably, a selection of nucleic acids comprising a same number of nucleotide bases has a substantially the same or similar melting temperature (Tₘ), which is useful when using nucleic acids as irrelevant binding pairs for an assay that captures multiple distinct analytes.

The nucleic acid of the invention may be used concurrently, for the discrete capture or immobilisation of a multiplicity of ligands, analytes, or other species, analyte/anti-analyte and anti-analyte/analyte/reporter species complexes, without non-specific hybridisation, from a mixture in solution, on a support matrix at specified capture zones for each ligand, analyte, or other species or complex where the nucleotide sequences can be of any length and where the length of the nucleic acid is not subject to the formation of stable secondary structure or dimers which may reduce hybridisation efficiency.

A first aspect of the invention provides an isolated nucleic acid comprising a repeated nucleotide sequence of at least two different nucleotide bases having a minimal repeating nucleotide sequence of three or more nucleotide bases, whereby said nucleic acid is characterised by:

i) lacking an internal secondary structure;

ii) capable of hybridising with a complementary nucleic acid; and

iii) does not hybridise with a non-complementary nucleic acid; under at least low stringency conditions.

In one embodiment, the at least two different nucleotide bases are grouped so that the repeating nucleotide sequence comprises a series of adjacent A or T nucleotide bases followed by a series of adjacent G or C nucleotide bases.

In another embodiment, the at least two different nucleotide bases are grouped so that the repeating nucleotide sequence comprises a series of adjacent G or C nucleotide bases followed by a series of adjacent A or T nucleotide bases.

Preferably, the isolated nucleic acid is selected from the group consisting of:

5′ TATGCGGCG TATGCGGCG 3′
5′ TTAAATGGC TTAAATGGC 3′
5′ TATTATCCCCCG TATTATCCCCCG 3′

Preferably, the isolated nucleic acid comprises a repeating nucleotide sequence of three to twelve nucleotide bases.

More preferably, the isolated nucleic acid comprises a repeating nucleotide sequence of six nucleotide bases.

In a second aspect the invention provides an isolated nucleic acid consisting essentially of a nucleotide sequence as defined by a formula selected from the group consisting of: I, II, Ia, IIa, Ib and IIb, wherein the respective formulas are as follows:

5′ Y₁[(X₁X₂X₃X₄X₅X₆)]ₙ Y₂3′ (I)

3′ Y₁[(X₁X₂X₃X₄X₅X₆)]ₙ Y₂5′ (II)

wherein:

(A) when X₁, X₂ and X₃ are either A or T

then X₄, X₅ and X₆ are either G or C; or

(B) when X₁, X₂ and X₃ are either G or C

then X₄, X₅ and X₆ are either A or T;

n≧2

Y₁ is selected from the group consisting of:

X₂ X₃ X₄ X₅ X₆

X₃ X₄ X₅ X₆

X₄ X₅ X₆

X₅ X₆

X₆ and zero

Y₂ is selected from the group consisting of:

X₁ X₂ X₃ X₄ X₅

X₁ X₂X₃ X₄

X₁X₂X₃

X₁X₂

X₁ and zero

5′ Y₁[(X₁X₂X₃X₄X₅)]ₙY₂3′ (Ia)

3′ Y₁[(X₁X₂X₃X₄X₅)]ₙY₂5′ (IIa)

wherein:

(A) when X₁, X₂ and X₃ are either A or T

then X₄ and X₅ are either G or C;

(B) when X₁, X₂ and X₃ are either G or C

then X₄ and X₅ are either A or T;

(C) when X₁ and X₂ are either A or T

then X₃, X₄ and X₅ are either G or C; or

(D) when X₁ and X₂ are either G or C

then X₃, X₄ and X₅ are either A or T;

n≧2

Y₁ is selected from the group consisting of:

X₂ X₃ X₄ X₅

X₃ X₄ X₅

X₄ X₅

X₅ and zero

Y₂ is selected from the group consisting of:

X₁ X₂ X₃ X₄

X₁ X₂X₃

X₁X₂

X₁

X₁ and zero

5′ Y₁[(X₁X₂X₃X₄)]ₙY₂3′ (Ib)

3′ Y₁[(X₁X₂X₃X₄)]ₙY₂5′ (IIb)

wherein:

(A) when X₁ and X₂ are either A or T

then X₃ and X₄ are either G or C;

(B) when X₁ and X₂ are either G or C

then X₃ and X₄ are either A or T;

n≧2

Y₁ is selected from the group consisting of:

X₂ X₃ X₄

X₃ X₄

X₄ and zero

Y₂ is selected from the group consisting of:

X₁ X₂ X₃

X₁ X₂

X₁ and zero

Preferably, n=2-20.

More preferably, n=2-6.

In one embodiment, the isolated nucleic acid of the second aspect is selected from the group consisting of:

5′ GC(TAACGC)₄T 3′;
5′ TT(CCCTTT)4CCCTT 3′;
5′ (TATGGC)₄TAT 3′;
5′ AACCG(TAACCG)₄T 3′;
5′ G(TTACCG)₄TT 3′;
5′ (ATTGGG)₆ 3′;
5′ GC(TACGC)₄T 3′;
5′ TT(CCCTT)₄CCCT 3′;
5′ (GCTA)₄ GCT 3′;
5′ ACC(TACC)₆T 3′;
5′ CCCTAA CCCTAACCCTAACCCTAACCCTAACCCTAA 3′;
5′ CGGAATCGGAATCGGAATCGGAATCGGAATCGGAAT 3′; and
5′ ATTCCGATTCCGATTCCG 3′

Preferably, the isolated nucleic acid of the second aspect is selected from the group consisting of:

| | | |
|---|---|---|
| 5′ (AAAGGG)ₙ 3′ | 5′ (AAAGGC)ₙ 3′ | 5′ (AAAGCG)ₙ 3′ |
| 5′(AAAGCC)ₙ 3′ | | |
| 5′ (AAACCC)ₙ 3′ | 5′ (AAACCG)ₙ 3′ | 5′ (AAACGC)ₙ 3′ |
| 5′(AAACGG)ₙ 3′ | | |
| 5′ (TTTGGG)ₙ 3′ | 5′ (TTTGGC)ₙ 3′ | 5′ (TTTGCG)ₙ 3′ |
| 5′(TTTGCC)ₙ 3′ | | |
| 5′ (TTTCCC)ₙ 3′ | 5′ (TTTCCG)ₙ 3′ | 5′ (TTTCGC)ₙ 3′ |
| 5′(TTTCGG)ₙ 3′ | | |
| 5′ (AATGGG)ₙ 3′ | 5′ (AATGGC)ₙ 3′ | 5′ (AATGCG)ₙ 3′ |
| 5′(AATGCC)ₙ 3′ | | |
| 5′ (AATCCC)ₙ 3′ | 5′ (AATCCG)ₙ 3′ | 5′ (AATCGC)ₙ 3′ |
| 5′(AATCGG)ₙ 3′ | | |
| 5′ (TTAGGG)ₙ 3′ | 5′ (TTAGGC)ₙ 3′ | 5′ (TTAGCG)ₙ 3′ |
| 5′(TTAGCC)ₙ 3′ | | |
| 5′ (TTACCC)ₙ 3′ | 5′ (TTACCG)ₙ 3′ | 5′ (TTACGC)ₙ 3′ |
| 5′(TTACGG)ₙ 3′ | | |
| 5′ (TAAGGG)ₙ 3′ | 5′ (TAAGGC)ₙ 3′ | 5′ (TAAGCG)ₙ 3′ |
| 5′(TAAGCC)ₙ 3′ | | |
| 5′ (TAACCC)ₙ 3′ | 5′ (TAACCG)ₙ 3′ | 5′ (TAACGC)ₙ 3′ |
| 5′(TAACGG)ₙ 3′ | | |
| 5′ (ATTGGG)ₙ 3′ | 5′ (ATTGGC)ₙ 3′ | 5′ (ATTGCG)ₙ 3′ |
| 5′(ATTGCC)ₙ 3′ | | |
| 5′ (ATTCCC)ₙ 3′ | 5′ (ATTCCG)ₙ 3′ | 5′ (ATTCGC)ₙ 3′ |
| 5′(ATTCGG)ₙ 3′ | | |
| 5′ (ATAGGG)ₙ 3′ | 5′ (ATAGGC)ₙ 3′ | 5′ (ATAGCG)ₙ 3′ |
| 5′(ATAGCC)ₙ 3′ | | |
| 5′ (ATACCC)ₙ 3′ | 5′ (ATACCG)ₙ 3′ | 5′ (ATAGGC)ₙ 3′ |
| 5′(ATACGG)ₙ 3′ | | |
| 5′ (TATGGG)ₙ 3′ | 5′ (TATGGC)ₙ 3′ | 5′ (TATGCG)ₙ 3′ |
| 5′(TATGCC)ₙ 3′ | | |
| 5′ (TATCCC)ₙ 3′ | 5′ (TATCCG)ₙ 3′ | 5′ (TATCGC)ₙ 3′ |
| 5′(TATCGG)ₙ 3′ | | |

The isolated nucleic acid of the first and second aspects may comprise derivatives of respective A, T, C or G nucleotide bases.

Preferably, two or more isolated nucleic acids comprising a same number of nucleotide bases are characterised by a Tₘ substantially identical or similar to each other.

The use of a nucleic acid comprising complementary sequences as discussed above in formulas (I), (II), (Ia), (IIa), (Ib) and (IIb) as irrelevant binding pairs for the capture of multiple analytes or analyte/anti-analyte complexes or for the immobilisation of various other species should meet with a number of criteria for an effective and efficient application of this capture system in any of these formats.

(a) Each nucleic acid in most cases should hybridise only with a nucleic acid comprising a complementary sequence thereto which is attached to a support matrix and with none of the other nucleic acids either attached to the support matrix or in solution. This high level of hybridisation specificity is critical to ensure that little or no cross-reactivity capture occurs and to ensure that cross-dimerisation between various nucleic acids in solution, which would minimise the sensitivity of the assay, is also minimised or does not occur.

(b) Pairs of nucleic acids comprising complementary sequences in most cases should also hybridise efficiently and with high specificity under low or relaxed stringency conditions as high stringency conditions may not be compatible with conditions needed for optimal analyte/anti-analyte association.

(c) Individual nucleic acids must have little or no stable secondary structure, such as hairpin loops, etc., or form stable dimers, particularly, at room temperature as this would significantly reduce the rate of hybridisation and hence the capture efficiency particularly with increasing length of the nucleic acid.

(d) Melting temperature, G-C content and nucleic acid length of each nucleic acid should at least be similar, preferably the same, as the melting temperature, G-C content and nucleic acid length of other nucleic acids used in an assay system to ensure that hybridisation occurs at similar, preferably the same, rate with similar, preferably the same, level of efficiency between complementary pairs of nucleotide sequences for a given hybridisation condition.

In one embodiment, the isolated nucleic acid of the first and second aspects may further comprise one or more linkers attached to or contiguous with any respective one or more nucleotide bases thereof.

The linker may be selected from the group consisting of: a substituted or unsubstituted alkyl group having one or more carbons, wherein the alkyl groups may be linear or branched; a substituted or unsubstituted aryl or aryl alkyl group; proteins and peptides, including linear and branched peptides; peptides comprising a lysine residue; nucleic acids, including oligonucleotides and primers; dendrimers or dendrimeric like molecules; polymers; oligomers comprising a plurality of units; and other linear polymeric materials.

In another embodiment, the isolated nucleic acid comprises a nucleotide sequence that is contiguous with a nucleotide sequence of the linker.

In yet another embodiment, one or more species is respectively attached to or is contiguous with the one or more linkers.

In a further embodiment, the isolated nucleic acid further comprises one or more species respectively attached to or contiguous with any one or more nucleotide bases thereof.

The isolated nucleic acid may be attached to or contiguous with an expressed protein.

A nucleic acid structure may comprise a structure defined by a formula selected from the group consisting of:

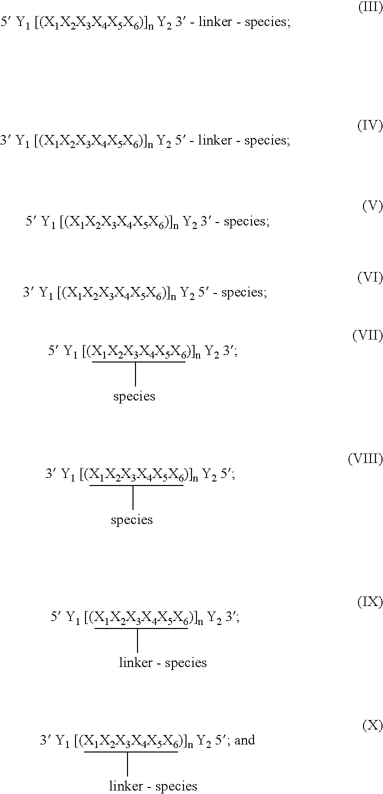

where in formulae (VII) to (X) the species may be attached to any one or more nucleotide bases thereof.

The isolated nucleic acid may be used as a linker for cross linking two or more species in solution, at a mixed interphase, at a solid—solid phase interface, or combination thereof, by hybridising a complementary binding pair of said isolated nucleic acids wherein each respective complementary isolated nucleic acid is attached to at least one of said two or more species.

The species may be selected from the group consisting of: antibodies, antibody fragments including a light chain fragment, antibody mimetics, antigens or parts thereof, antigen mimetics, enzymes, proteins, peptides, organic molecules, haptens, pharmaceutical compounds, dendrimers and dendrimeric-like molecules, coloured dendrimers, beads, coloured beads, latex beads, microparticles or other coloured polymeric or branched materials, gold microparticles, reporter molecules, fluorochromes (or fluorescent compounds), dyes, metal chelates, radioactive isotopes, nucleic acids including oligonucleotides and nucleic acid amplification products including PCR products, RNA, DNA, PNA and synthetic oligonucleotides.

In one embodiment, one or more isolated nucleic acid(s) comprising a nucleotide sequence 5′ ATTCCGATTCCGATTCCG 3′ are attached to one or more coloured latex bead(s).

In another embodiment, one or more species is respectively attached to one or more nucleotide base(s) internal of terminal ends of said isolated nucleic acid.

Preferably, the one or more species comprises multiple reporter molecules.

More preferably, the isolated nucleic acid comprising multiple reporter molecules is used as a signal reagent for detecting one or more target(s).

A 3′ or 5′ end of the isolated nucleic acid may be immobilised to a support such that the isolated nucleic acid may be capable of hybridising with one or more target(s).

The isolated nucleic acid may comprise a nucleotide sequence 5′CCCTAA CCCTAACCCTAACCCTAACCCTAACCCTAA 3′ or 5′CGGAATCGGAATCGGAATCGGAATCGGAATCGGGAAT 3′.

The support may be selected from the group consisting of: a membrane, a microarray chip, and surface of a dish, well, insoluble support matrix, microtiter plate or microparticle.

The isolated nucleic acids may be immobilised to a support and used with a lateral flow assay.

In a third aspect, the invention provides an array comprising:

A) a support; and

B) one or more isolated nucleic acids of claim 1 or claim 7 immobilised to the support.

In one embodiment, the support is selected from the group consisting of: glass, plastic, polymeric material and a gel.

Preferably, the array is a microarray or a multi-well plate.

In another embodiment, the support is a labelled microparticle, including a dendrimer.

Preferably, the labelled microparticle is a coloured microparticle.

Preferably, the one or more nucleic acid(s) comprise a same nucleotide sequence.

Preferably, the array is characterised as a generic array in solution.

In a fourth aspect, the invention provides a method for capturing one or more target(s) which includes the step of immobilising one or more isolated nucleic acids of the first or second aspect to a support wherein each immobilised isolated nucleic acid is capable of hybridising with a complementary nucleic acid.

The one or more targets may include analytes or other species as described above.

In a fifth aspect, the invention provides a method of linking two or more species, the method including the step of respectively attaching an isolated nucleic acid of the first or second aspect to said respective two or more species, wherein said isolated nucleic acids are capable of forming a complementary binding pair.

In a sixth aspect, the invention provides a method of making a signal reagent, said method including the step of attaching one or more reporter molecules to an isolated nucleic acid of the first or second aspect.

### BRIEF DESCRIPTION OF THE FIGURES

FIG. 1 shows an example of a lateral flow assays comprising nucleic acid hybridization components as receptor-binders.

FIG. 2A shows a universal lateral flow filter strip suitable for detection of up to four analytes.

FIG. 2B shows analytes in an antibody and antigen lateral flow immunoassay test.

FIG. 2C shows examples of reagents required for the filter strip shown in FIG. 2A for detection of IgA, IgG and IgM antibodies to an antigen and an antigen in a serum sample.

FIG. 2D shows immunoassays complexes captured on a filter strip.

FIG. 2E shows appearance of positive IgG, IgM, IgA and antigen test results on a filter strip, for example as shown in FIG. 2D.

FIG. 3A shows nucleic acid analytes in a lateral flow assay.

FIG. 3B shows examples of reagents required for the filter strip shown in FIG. 2A for identification of four different nucleic acids having target sequences, F1, F2, F3 and F4.

FIG. 3C shows a generic representation of hybridization events in a nucleic acid lateral flow assay.

FIG. 3D shows a test strip as it would appear if nucleic acid analytes F1, F2, F3 and F4 were all detected in amplified sample, for example as in FIG. 3C.

FIG. 4A shows a means of attaching a receptor nucleic acid to a filter to perform a nucleic acid rapid lateral flow assay.

FIG. 4B shows reagents used for a nucleic acid detection assay in a bottle.

FIG. 5 shows hybridization events in a nucleic acid detection assay using a coloured microparticle as a component of a detector agent.

FIG. 6 shows steps involved in a nucleic acid detection assay by rapid lateral flow.

FIG. 7 shows application of receptor-binder nucleic acids in biochips.

FIG. 8 shows sub-arrays applied to multi-well plates.

FIG. 9 shows an example of a microarray of 25 sub-wells, in either a microchip or microplate format for use in nucleic acid or protein applications.

FIG. 10 shows an example of two species linked by complementary binding of two complementary nucleic acids of the invention.

### DESCRIPTION OF PREFERRED EMBODIMENTS

Definitions

Unless defined otherwise, all technical and scientific terms used herein have the meaning as commonly understood by those of ordinary skill in the art to which the invention belongs. Although any methods and materials similar or equivalent to those described herein can be used in the practice or testing of the present invention, preferred methods and materials are described. For the purpose of the present invention, the following terms are defined below.

The term “linear” as used herein means a molecule, in particular a nucleic acid, which has a stable linear configuration excluding elements of secondary structure such as hairpin loops which may form from self-hybridisation.

The term “ligand” or “reactive ligand” refers to one member of a binding pair, which may have been incorporated into the analyte and may include but is not limited to antibodies, lectins, receptors, binding proteins or chemical agents.

The term “binding pair” or “complementary binding pair” includes any of the class of immune-type binding pairs, such as antigen/antibody or hapten/anti-hapten systems; and also any of the class of nonimmune-type binding pairs, such as biotin/avidin; biotin/streptavidin; folic acid/folate binding protein; complementary nucleic acids capable of hybridisation; protein A or G and immunoglobulins; and binding pairs which form covalent bonds, such as sulfhydryl reactive groups including maleimides and haloacetyl derivatives, and amine reactive groups such as isotriocyanates, succinimidyl esters and sulfonyl halides.

“Irrelevant binding pair” refers to a binding pair which is independent of the binding specificity of an analyte/anti-analyte interaction of interest.

The term “nucleic acid” as used herein designates single or double stranded mRNA, RNA, cRNA and DNA, said DNA inclusive of cDNA and genomic DNA.

“Oligonucleotide” herein refers to a nucleic acid comprising less than 150 contiguous nucleotides. The nucleotides can be joined by a variety of intersubunit linkages, including, but not limited to, natural phosphodiester bonds, phosphomono (Eckstein, F., 1985, Ann. Rev. Biochem., 54 367; Cohen, J. S., 1993, in Antisense Research and Applications, Crooke, T. S., et al., Eds., CRC Press, Boca Raton, Fla., pp. 205-221) or dithioate (Marshall, W. S., et al., 1993, Science, 259, 1564), methylphosphonates (Miller, P. S., 1991, Biotechnology, 9 358), or phosphodiester amidates (Letsinger, R. L., 1988, J. Am. Chem. Soc., 110 4470) all herein incorporated by reference. Further, “oligonucleotide” includes modifications, known to one skilled in the art, to a sugar backbone (e.g., ribose or deoxyribose subunits), sugar (e.g., 2′ substitutions), base, and 3′ and 5′ termini thereof. “Oligodeoxyribonucleotide” include such modifications, such as, 2′ sugar substitutions of flourine.

It will be appreciated that the nucleic acid or oligonucleotide backbone instead of comprising sugar groups linked by phosphodiester bridges, etc., may comprise of a polyamide backbone in a similar manner to peptide nucleic acids (PNAs) as described in International Patent Publication No. WO 92/20702 herein incorporated by reference. In this patent PNAs are described as compounds comprising a polyamide backbone bearing a plurality of ligands such as naturally occurring nucleotide bases attached to a polyamide backbone and consists of N-(2-aminoethylglycine units to which nucleotide bases are attached, is able to hybridise to complementary oligonucleotides to form natural PNA-nucleic acid complexes (Egholm, et al., 1993, Nature, 365, 566) herein incorporated by reference.

It will also be appreciated that the backbone of the oligonucleotide may comprise peptide-like units as well as units of sugar groups linked by phosphodiester bridges, optionally substituted with other groups such as phosphorothioates or methylphosphonates.

A “probe” may be a single or double-stranded nucleic acid, for example an oligonucleotide, suitably labeled for the purpose of detecting a nucleic acid having complementary sequences by molecular hybridisation, for example by Northern or Southern blotting.

“Nucleoside” is defined herein as a pentose sugar (a ribose, deoxyribose, or modification thereof) bound to a base capable forming hydrogen bonds (typically a purine or pyrimidine).

A “base” is defined herein to include (i) typical nucleotide bases for example DNA and RNA bases (uracil, thymine, adenine, guanine, and cytosine), and (ii) modified bases or base analogs (e.g., 5-methyl-cytosine, 5-bromouracil, or inosine). A base analogue is a chemical whose molecular structure mimics that of a typical DNA or RNA base.

The term “adenine” or “A” as used herein or “guanine” or “G” as used herein, means the normal adenine or guanine purine structure as well as adenine or guanine derivatives which include any inert substituent on the purine ring which does not detract from the biological activity of adenine or guanine. Examples of derivatives are hypoxanthine and xanthine.

The term “thymine” or “T” as used herein or “cytosine” or “C” as used herein, means the normal thymine or cytosine pyrimidine structure as well as thymine derivatives which include any inert substituent on the pyrimidine ring which does not detract from the biological activity of thymine or cytosine. Examples of such derivatives are uracil or orotic acid.

A “reporter probe” or “reporter agent” also refers to a “signal reagent” which may be used for detecting a target, for example an analyte. A signal reagent may comprise a nucleic acid having one or more reporter molecules, for example coloured bead or dendrimer.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

In DNA, complementary bases are:

(i) A and T; and

(ii) C and G.

In RNA, complementary bases are:

(i) A and U; and

(ii) C and G.

In RNA-DNA hybrids, complementary bases are:

(i) A and U;

(ii) A and T; and

(iii) G and C.

Modified purines (for example, inosine, methylinosine and methyladenosine) and modified pyrimidines (thiouridine and methylcytosine) may also engage in base pairing. Hybridise and hybridisation may also refer to pairing between complementary modified nucleic acids for example PNA and DNA, and PNA and RNA respectively.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to “low stringency conditions” includes and encompasses:—

(i) from at least about 1% v/v to at least about 15% v/v formamide and from at least about 1 M to at least about 2 M salt for hybridisation at 42° C., and at least about 1 M to at least about 2 M salt for washing at 42° C.; and

(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

“Medium stringency conditions” include and encompass:—

(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and

(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

“High stringency conditions” include and encompass:

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 680C for about 20 minutes.

In general, the Tₘ of a duplex DNA decreases by about 1° C. with every increase of 1.7% in the number of mismatched bases. Description of mismatched bases and Tₘ of a duplex DNA is provided in Anderson, 1999, “Nucleic Acid Hybridisation”, Pub. BIOS Scientific Publishers Ltd, Springer, Glasgow, UK.

“Relaxed conditions” is herein defined as hybridisation conditions permitting nucleic acid duplexes to form even though mismatched nucleotide bases exist in the respective nucleic acids (eg. the hybridising nucleic acids are related, but are not identical). High salt concentrations and low temperatures of washing permit imperfectly matched nucleic acids to hybridise. Relaxed conditions are further described in Anderson, 1999, In Nucleic Acid Hybridisation, Rickwood, D. ed. BIOS Scientific Publishers Ltd., Oxford, UK, herein incorporated by reference.

Notwithstanding the above, stringent conditions are well known in the art, such as described in Chapters 2.9 and 2.10 of Ausubel et a/., supra, which are herein incorporated by reference. A skilled addressee will also recognize that various factors can be manipulated to optimize the specificity of the hybridization. Optimization of the stringency of the final washes can serve to ensure a high degree of hybridization.

The term “linker” as used herein means any spacer group which can be used to link various entities as described above. Any suitable linking group can be used which may include substituted or unsubstituted alkyl groups having from one to twenty, or more preferably one to six carbons, and wherein the alkyl groups may be linear or branched. Linkers may also comprise substituted or unsubstituted aryl or aryl alkyl groups. Thus for example the linking group may be variable comprising a single methylene or a plurality of methylene groups. The linking group may also comprise peptides or branched peptides inclusive of lysine. The linking group may also comprise dendrimers or dendrimeric like molecules; polymers; oligomers which comprise a plurality of units, typically repeating units, for example nucleic acids; and linear polymeric material.

“Species” includes antibodies, antibody fragments such as the light chain fragment, antibody mimetics, antigens or parts thereof, antigen mimetics, enzymes, peptides, organic molecules, haptens, pharmaceutical compounds, dendrimers and dendrimeric-like molecules, coloured dendrimers, beads, latex beads, coloured beads, microparticles or other coloured polymeric or branched materials, gold microparticles, reporter molecules, fluorochromes (or fluorescent compounds), dyes, metal chelates, radioactive isotopes, nucleic acid amplification products such as PCR products, RNA, DNA, PNA, synthetic oligonucleotides, having a definition as discussed hereinafter. Species may be attached, for example conjugated, to an oligonucleotide.

A “support” includes a support matrix and solid support such as a dish, tray, plate, multi-well plate, microtiter plate, bead, dendrimer and micro-chip. Support also refers to bibulous material including silica gel and other useful gels, cellulosic beads, glass and glass fibres, filter paper and natural and synthetic membranes such as nitrocellulose and nylon membranes. A support may comprise glass, plastic or other polymeric material, gel elements fixed on a solid support.

Throughout this specification, unless the context requires otherwise, the words “comprise”, “comprises” and “comprising” will be understood to imply the inclusion of a stated integer or group of integers but not the exclusion of any other integer or group of integers.

In order that the invention may be readily understood and put into practical effect, particular preferred embodiments will now be described by way of the following non-limiting examples.

The following sequences may serve as examples of the invention.

| | |
|---|---|
| Example A: | 5′ GC(TAACGC)₄T 3′ |
| Example B: | 5′ TT(CCCTTT)₄CCCTT 3′ |
| Example C: | 5′ (TATGGC)₄TAT 3′ |
| Example D: | 5′ AACCG(TAACCG)₄T 3′ |
| Example E: | 5′ G(TTACCG)₄TT 3′ |
| Example F: | 5′ (ATTGGG)₆3′ |
| Example G: | 5′ GC(TACGC)₄T 3′ |
| Example H: | 5′ TT(CCCTT)₄CCCT 3′ |
| Example I: | 5′ (GCTA)₄GCT 3′ |
| Example J: | 5′ ACC(TACC)₆T 3′ |

The following sequences may serve as examples of an embodiment of the invention in relation to a heximer (6-mer) nucleic acid which may be repeated. Examples below show nucleotide sequence heximers comprising three nucleotides which may be either A or T at a 5′ end and adjacent thereto are three nucleotides which may be either G or C at a 3′ end. Alternatively or in addition, a heximer may comprise three nucleotides which may be either G or C at a 5′ end and adjacent thereto three nucleotides which may be either A or T at a 3′ end. These nucleic acids may be flanked on either or both sides by additional nucleic acid as described herein. Complementary nucleotide sequences shown below may be used as binding pairs thereof.

| | | |
|---|---|---|
| 5′ (AAAGGG)ₙ 3′ | 5′ (AAAGGC)ₙ 3′ | 5′ (AAAGCG)ₙ 3′ |
| 5′(AAAGCC)ₙ 3′ | | |
| 5′ (AAACCC)ₙ 3′ | 5′ (AAACCG)ₙ 3′ | 5′ (AAACGC)ₙ 3′ |
| 5′(AAACGG)ₙ 3′ | | |
| 5′ (TTTGGG)ₙ 3′ | 5′ (TTTGGC)ₙ 3′ | 5′ (TTTGCG)ₙ 3′ |
| 5′(TTTGCC)ₙ 3′ | | |
| 5′ (TTTCCC)ₙ 3′ | 5′ (TTTCCG)ₙ 3′ | 5′ (TTTCGC)ₙ 3′ |
| 5′(TTTCGG)ₙ 3′ | | |
| 5′ (AATGGG)ₙ 3′ | 5′ (AATGGC)ₙ 3′ | 5′ (AATGCG)ₙ 3′ |
| 5′(AATGCC)ₙ 3′ | | |
| 5′ (AATCCC)ₙ 3′ | 5′ (AATCCG)ₙ 3′ | 5′ (AATCGC)ₙ 3′ |
| 5′(AATCGG)ₙ 3′ | | |
| 5′ (TTAGGG)ₙ 3′ | 5′ (TTAGGC)ₙ 3′ | 5′ (TTAGCG)ₙ 3′ |
| 5′(TTAGCC)ₙ 3′ | | |
| 5′ (TTACCC)ₙ 3′ | 5′ (TTACCG)ₙ 3′ | 5′ (TTACGC)ₙ 3′ |
| 5′(TTACGG)ₙ 3′ | | |
| 5′ (TAAGGG)ₙ 3′ | 5′ (TAAGGC)ₙ 3′ | 5′ (TAAGCG)ₙ 3′ |
| 5′(TAAGCC)ₙ 3′ | | |
| 5′ (TAACCC)ₙ 3′ | 5′ (TAACCG)ₙ 3′ | 5′ (TAACGC)ₙ 3′ |
| 5′(TAACGG)ₙ 3′ | | |
| 5′ (ATTGGG)ₙ 3′ | 5′ (ATTGGC)ₙ 3′ | 5′ (ATTGCG)ₙ 3′ |
| 5′(ATTGCC)ₙ 3′ | | |
| 5′ (ATTCCC)ₙ 3′ | 5′ (ATTCCG)ₙ 3′ | 5′ (ATTCGC)ₙ 3′ |
| 5′(ATTCGG)ₙ 3′ | | |
| 5′ (ATAGGG)ₙ 3′ | 5′ (ATAGGC)ₙ 3′ | 5′ (ATAGCG)ₙ 3′ |
| 5′(ATAGCC)ₙ 3′ | | |
| 5′ (ATACCC)ₙ 3′ | 5′ (ATACCG)ₙ 3′ | 5′ (ATACGC)ₙ 3′ |
| 5′(ATACGG)ₙ 3′ | | |
| 5′ (TATGGG)ₙ 3′ | 5′ (TATGGC)ₙ 3′ | 5′ (TATGCG)ₙ 3′ |
| 5′(TATGCC)ₙ 3′ | | |
| 5′ (TATCCC)ₙ 3′ | 5′ (TATCCG)ₙ 3′ | 5′ (TATCGC)ₙ 3′ |
| 5′(TATCGG)ₙ 3′ | | |

Although a nucleic acid of the invention preferably comprises a nucleotide sequence as defined by formulas I, II, Ia, IIa, Ib and IIb, other nucleic acids are contemplated by the inventors that comprise a repeated nucleotide sequence characterised by

i) lacking an internal secondary structure;

ii) capable of hybridising with a complementary nucleic acid; and

iii) does not hybridise with a non-complementary nucleic acid;

under at least low stringency conditions.

Such nucleic acids may include, for example, a repeating nucleic acid unit comprising a series of A or T nucleotides of any suitable length located at a 5′ end adjacent a series of G or C nucleotides of any suitable length located at a 3′ end, or vice versa. Some examples of such nucleic acids include the following:

5′ TATGCGGCG TATGCGGCG 3′
5′ TTAAATGGC TTAAATGGC 3′
5′ TATTATCCCCCG TATTATCCCCCG 3′

It will be appreciated that nucleic acids comprising a nucleotide sequence as described herein may provide pre-selection of a nucleotide sequence which may have the characteristics i), ii) and iii) above.

It will be appreciated that methods for generating and purifying synthetic oligonucleotides, including the above examples, are common and well known in the art (Rushdi, A., 1993, in DNA Probes, Keller, G. H. and Manak, M. M., Eds., Stockton Press, N.Y., USA, pp. 69-136; Ausbel et al., 1990, Current Protocols in Molecular Biology, Supplement 9, 2, 11), herein incorporated by reference.

A nucleic acid of the present invention may be attached to support matrices through a number of well-known methodologies as practised in the art. For example, the nucleic acid may be directly immobilised on nitrocellulose membranes or other suitable supports by ultraviolet irradiation, baking, capillary transfer or vacuum transfer (Kalachikov, S. M., et al., 1992, Biorog. Khim., 18, 52; Nierzwicki-Bauer, et al., 1990, Biotechniques, 9, 472), herein incorporated by reference. A nucleic acid may also be directly immobilised onto nitrocellulose membrane through the incorporation of a poly-T tail at one end which allows the nucleic acid to be attached via UV fixation (Brown and Anthony, 2000, Jour. Microbiol Methods 42 203).

The nucleic acid may also be attached to support matrices via covalent bond formation. For example, the nucleic acid may be derivatised with a thiol group and reacted with sulfhydryl reactive groups such as maleimides and haloacetyl derivatives present on the support matrix or it may be derivatised with an amine group and reacted with amine reactive groups, on the support matrix, such as isothiocyanates, succinimidyl esters and sulfonyl halides (Ghosh, S. S., et al., 1993, Nucleic Acids Research, 21, 1819), herein incorporated by reference. Solid supports, such as microtitre plates, pre-activated with either amine or sulfhydryl reactive groups are commercially available from suppliers such as Pierce (Rockford, Ill.), Exiqon A/S (Denmark), or Corning (New York, N.Y.). Alternatively, the nucleic acid may be synthesised directly onto the support matrix. For example, on filter membranes as described in U.S. Pat. No. 4,923,901 or using light-directed chemical synthesis for the generation of high density probe arrays on microchips (Lipshutz., et al., 1995, BioTechniques, 19, 442), both of which are incorporated herein by reference. Oligonucelotides may be directly synthesised on beads using standard or modified linkers and using standard phosphoroamidite chemistry either manually or via an automated synthesiser which are well known and practiced in the art. Nucleic acids may be derivatised with metal coordinating groups such as described in International Patent Publication No. WO 98/00435 or through the introduction of metal chelating groups such as EDTA using specially modified phosphoroamidites such as EDTA-C2-dT-CE phosphoroamidite (Glen Research), herein incorporated by reference and attached, via metal chelate co-ordination, to support matrixes derivatised with metal chelating groups. For example, metal chelate microtitre plates and microparticles (Qiagen), or metal chelate filters (International Patent Publication No. WO 70012).

The nucleic acid may be indirectly attached to a support matrix by the interaction of an incorporated ligand with a previously immobilised capture agent. For example, the nucleic acid may be derivatised with a biotin and captured on a streptavidin or avidin or anti-biotin antibodies immobilised on the support matrix (Viscidi, R. P., et al., 1989, Journal of Clinical Microbiology, 27, 120), herein incorporated by reference. Ligands may be incorporated into the nucleic acid either at the 3′ end, the 5′ end or at any point between the 3′ and 5′ ends of the nucleic acid. Other methods of indirect nucleic acid attachment include the conjugation of the nucleic acid to a carrier molecule capable of binding to a support matrix through non-covalent bond type interactions such as hydrophobic or ionic charge based interactions. Examples of such carrier molecules include, but are not limited to, bovine serum albumin, polylysine, polyethylene glycol, dendrimers or other branched polymeric materials, microparticles.

It will further be appreciated that nucleic acid sequences of the invention may be linked to other species at either the 3′ or 5′ ends or anywhere along the nucleic acid sequence through a modified base as is commonly practised in the art. Examples of nucleic acid modifications and general conjugation procedures may be found in the following references incorporated herein by reference. Rushdi, A., 1993, in DNA Probes, Keller, G. H. and Manak, M. M., Eds., Stockton Press, N.Y., USA, pp. 69-136; Simmonds, A. and Cunningham, M., 1998, in Bioconjucation—Protein Coupling Techniques for the Biomedical Sciences, Aslam, M. and Dent, A., Eds., Macmillan References Ltd., London, UK, pp. 483-503; Hermanson, G. T., 1996, in Bioconjugate Techniques, Hermanson, G. T., Ed., Academic Press, NY, pp. 639-671 and references incorporated therein.

### Experimental

Oligonucleotides

Oligonucleotides were obtained from a commercial supplier (Auspep, Pty Ltd., Australia), were prepared using standard phosphoroamidite chemistry on an automated oligonucleotide synthesiser and were supplied in a lyophilised form. Oligonucleotides used for 5′ end derivatisation were supplied on the resin with the trityl group at the 5′ end remaining intact. The trityl group was manually cleaved off with 3% trichloroacetic acid in dichloromethane before manually coupling with [S trityl-6-mercaptohexyl)-(2-cyanoethyl)-(N,N-diisopropyl)] phosphoroamidite in the presence of tetrazole.

### EXAMPLE 1

Preparation of Maleimido Derivatised Microparticles

Fifty microliters of blue coloured latex microparticles (0.06 μm) at a 10% solid in water were diluted with 50 μL of 200 mM sodium acetate buffer pH 5.0. To this was then added a mixture of 1-Ethyl-3-(3-dimethylaminopropyl) carbodiimide (EDC) (0.17 μmol, 31 μg) and N-(β-maleimidopropionic acid) hydrazide.TFA (BMPH) (0.15 μmol, 45 μg) in 50 μL of 100 mM sodium acetate buffer, pH 5.0. The reaction mixture was left to stand at room temperature for 2 hrs and then dialysed against 50 mM phosphate buffer, pH 7.0 (3×1L).

### EXAMPLE 2

Preparation of Thiol Derivatised Oligonucleotides

Oligonucleotides were prepared with a 5′ thiol group using [S trityl-6-mercaptohexyl)-(2-cyanoethyl)-(N,N-diisopropyl)] phosphoroamidite and purified by reversed-phase HPLC. The trityl group was then removed with silver nitrate with the excess silver nitrate being removed with dithiothritol (DTT). The DTT group was removed by HPLC before reacting the 5′ thiol oligonucleotide with the maleimido derivatised microparticles.

### EXAMPLE 3

Preparation of Oligonucleotide-Microparticle Conjugate

One hundred μg of 5′ thiol oligonucleotide were incubated with 5 mg of maleimido derivatised microparticles in 50 mM phosphate buffer, pH 7.0 (0.15 mL) at room temperature overnight. The conjugate was then dialysed against 0.8 M NaCl solution (3×1L) to remove unreacted excess oligonucleotide.

### EXAMPLE 4

Immobilisation of Oligonucleotide Onto Nitrocellulose Membranes

A lateral flow strip format consisting of a nitrocellulose membrane with a glass fibre absorbent pad at each end, was spotted with two different oligonucleotide sequences. The first spot comprised an oligonucleotide of a non-complementary sequence to the oligonucleotide sequence conjugated to the coloured microparticle and the second spot comprised oligonucleotide of a complementary sequence to the conjugated oligonucleotide to the coloured microparticle. Both sequences were made up to a concentration of 1 μL/mL of deionised water and each spot was 0.5 μL in volume. The oligonucleotides were fixed to the nitrocellulose membrane by exposure to UV light for 15 min.

It will be appreciated that nucleic acids of the invention may be used with a membrane other than nitrocellulose, for example any bibulous absorbent solid material which may be formed as a wicking material which allows for capillary transport of liquids. A membrane is one form of a support.

Bibulous solid material include cellulose particles, silica gel and other useful gels, cellulosic beads, glass fibres or filter paper. Preferably, the wicking material is porous and can be moulded into complex sizes and shapes from thermoplastic polymers including light density polyethylene, ultra high molecular weight polyethylene, polyproylenen, polyvinylidene fluoride, polytetrafluoroethylene, nylon, polyethersulfone and ethyl vinyl acetate. Reference may be made to U.S. Pat. No. 4,552,839, incorporated herein by reference, which relates to analyte detection in a particle containing medium by contacting the medium with bibulous or wicking material wherein particles are concentrated at a position adjacent to the contact site by wicking of particles past the position and a signal associated with these particles can be used to measure the presence of the analyte.

### EXAMPLE 5

Assay Procedure

Coloured microparticles conjugated to oligonucleotide were placed in a solution of standard hybridisation buffer (SHB) consisting of 2 g/L of sodium dodecyl sulphate and 50 g/L of polyethylene glycol in 5× standard saline-citrate (SSC). The stock coloured microparticle conjugate was generally diluted by 10-fold for the assay. The rapid strip with the spotted oligonucleotide fixed on the nitrocellulose portion of the strip was dropped into a test tube (10×75 mm) with 100 μL of the diluted coloured microparticle conjugate placed at the bottom of the tube. The conjugate then immediately migrated up the strip to produce a visible colour change, within 5-10 min., at the spot where oligonucleotide complementary to the oligonucleotide sequence conjugated to the coloured microparticle had being immobilised. No colour change was observed at the spot where the immobilised oligonucleotide sequence was not complementary to the conjugated oligonucleotide sequence.

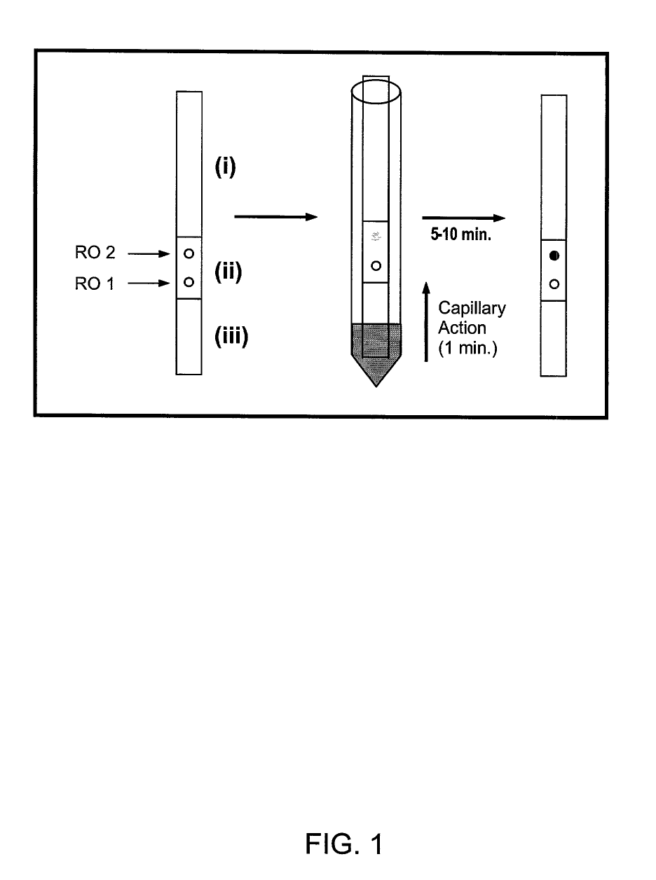

FIG. 1 shows results. (i) and (iii) represent the absorbent pad on the strip, (ii) is the nitrocellulose membrane part of the strip onto which receptor oligonucleotide 1 (RO1) and receptor oligonucleotide 2 (RO2) have been immobilised, and (iii) represents the part of the strip which is dipped into the conjugate buffer in the running tube. As the buffer flows upwards through the strip, an oligonucleotide conjugated to coloured microparticles hybridises to its complementary sequence, RO2, forming a coloured spot on the strip. As the microparticle does not carry oligonucleotides complementary to RO1, no hybridization between RO1 and this oligonucleotide occurs and no coloured spot appears on RO1.

Oligonucleotide sequences immobilised on the nitrocellulose membrane:

RO1 = 5′ CCCTAA CCCTAACCCTAACCCTMCCCTAACCCTAA 3′
RO2 = 5′ CGGAATCGGAATCGGAATCGGAATCGGAATCGGAAT 3′

Oligonucleotide sequence conjugated to blue latex beads 5′ ATTCCGATTCCGATTCCG 3′

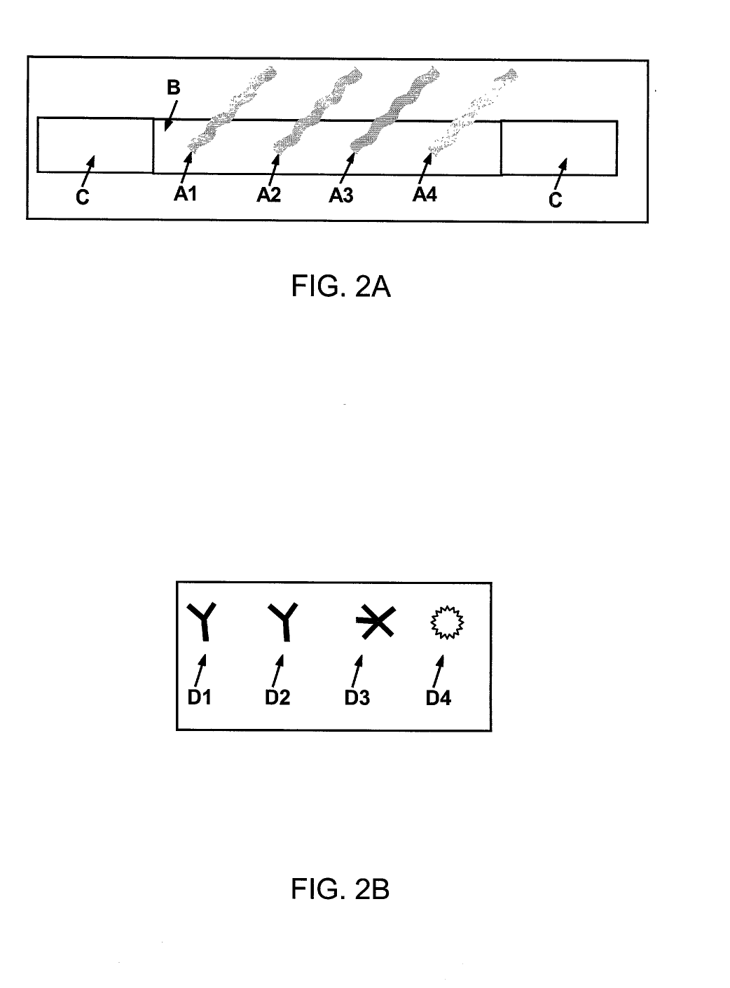

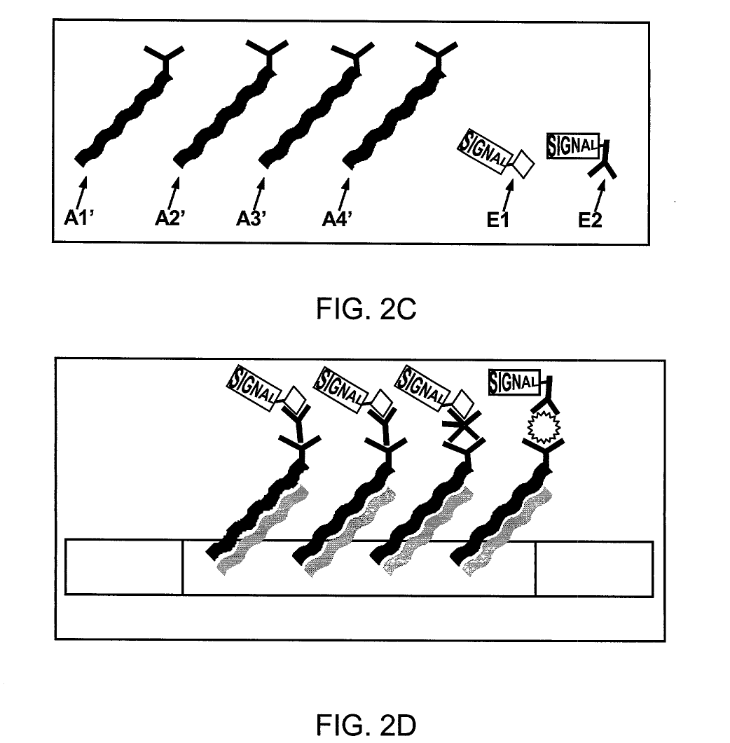

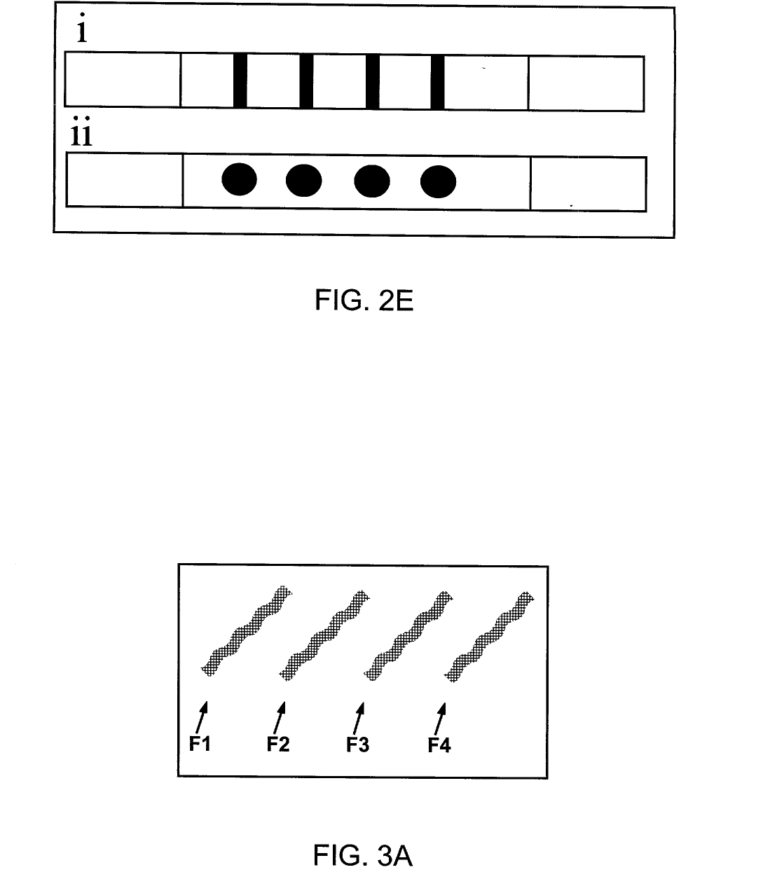

FIG. 2A shows a universal lateral flow filter strip suitable for detection of up to four different analytes, comprising filter strip B with glass fibre absorbant pads C located at opposite ends of filter strip B. Sprayed across or spotted on the filter strip at discrete intervals are four different receptor or capture oligonucleotide sequences A1, A2, A3 and A4.

FIG. 2B shows analytes in an antibody and antigen lateral flow immunoassay test. This is an example of analytes in a serum sample that might be captured in an immunoassay lateral flow system. D1 is IgA antibody in human serum. D2 is IgG antibody in human serum. D3 is IgM antibody in human serum. D4 is viral antigen present in human serum.

FIG. 2C shows examples of reagents required for the filter strip shown in FIG. 2A for the detection of IgA, IgG and IgM antibodies to an antigen and an antigen in a serum sample. A1′ is an oligonucleotide with a complementary sequence to A1 conjugated to anti-human IgA antibody. A2′ is an oligonucleotide with a complementary sequence to A2 conjugated to anti-human IgG antibody. A3′ is an oligonucleotide with a complementary sequence to A3 conjugated to anti-human IgM antibody. A4′ is an oligonucleotide with a complementary sequence to A4 conjugated to antibody specific for an antigen such as a viral antigen. El is detector agent comprising an antigen conjugated to a signal system such as a coloured microparticle or colloidal gold. E2 is a detector agent comprising a signal system conjugated antibody specific for the same antigen as the antibody conjugated in D′.

FIG. 2D shows immunoassays complexes captured on a filter strip. Shown are A1′, A2′, A3′ and A4′ carrying complexed analytes and detector agents, each of which are captured onto a filter strip via hybridisation to A1, A2, A3 and A4.

FIG. 2E shows appearance of positive IgG, IgM, IgA and antigen test results on a filter strip. FIG. 2E shows a strip as it would appear if IgA, IgG, and IgM antibody to an antigen of interest was detected in a sample, when an antigen was also detected in the sample. 2E(i) shows the appearance of a positive strip if oligonucleotides A1, A2, A3, & A4 were striped onto the filter strip. 2E(ii) shows the appearance of a positive strip if oligonucleotides A1, A2, A3, & A4 were spotted onto the filter strip.

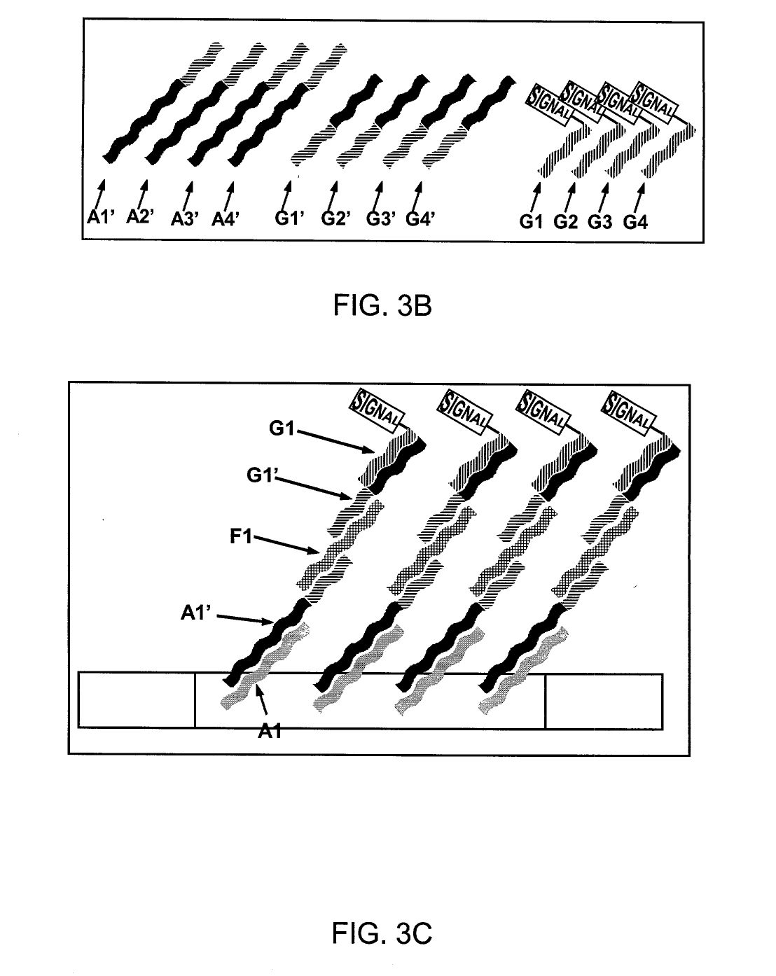

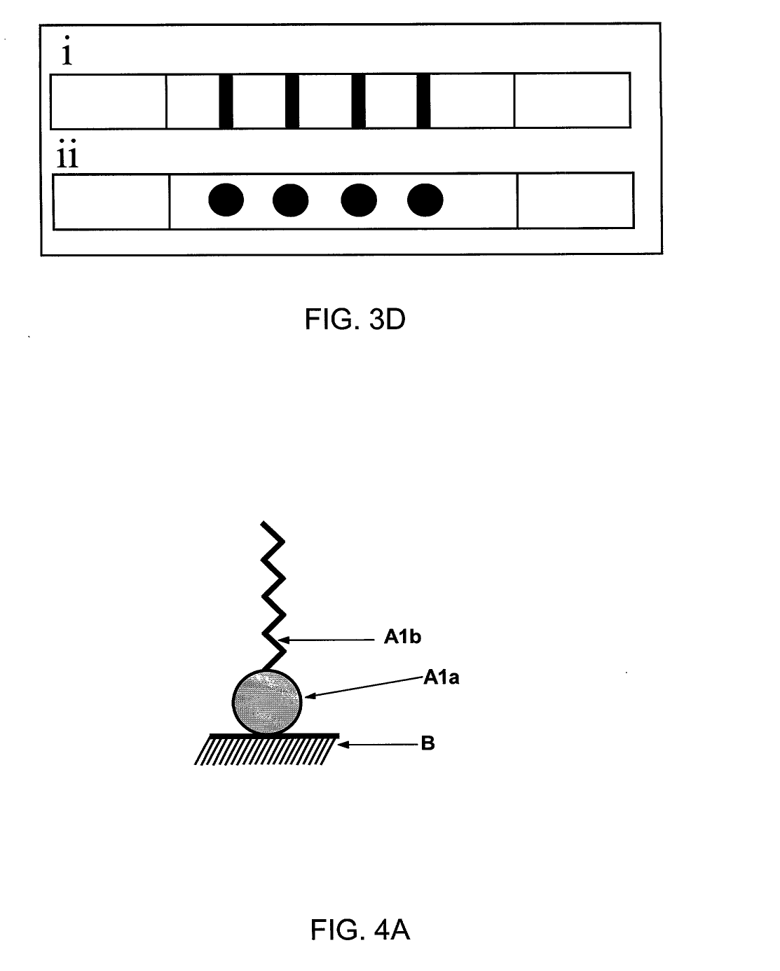

FIG. 3A shows nucleic acid analytes in lateral flow assays. FIG. 3A shows amplified nucleic acid analytes that would be captured in the lateral flow system to detect nucleic acid targets. F1 is a first amplified nucleic acid comprising a target nucleic acid sequence. F2 is a second amplified nucleic acid comprising a second target nucleic acid sequence. F3 is a third amplified nucleic acid comprising a target nucleotide sequence. F4 is a fourth amplified nucleic acid comprising a target nucleic acid sequence.

FIG. 3B shows examples of reagents required for the filter strip shown in FIG. 2A for identification of four different nucleic acids targets, F1, F2, F3 and F4 which have been amplified, for example by polymerase chain reactions, individual or multiplexed or similar process or biological amplification by culture of a virus, bacterium, parasite, yeast or fungus. A1′ is an oligonucleotide bridge with a complementary sequence to A1 contiguous with a first nucleic acid probe sequence that will hybridise with a unique sequence within F1. A2′ is an oligonucleotide bridge with complementary sequence to A2 contiguous with a second nucleic acid probe sequence that will hybridise with a unique sequence within F2. A3′ is an oligonucleotide bridge with complementary sequence to A3 contiguous with a third nucleic acid probe sequence that will hybridise with a unique sequence within F3. A4′ is an oligonucleotide bridge with complementary sequence to A4 contiguous with a fourth nucleic acid probe sequence that will hybridise with a unique sequence within F4.

G1 is detector agent comprising an oligonucleotide conjugated to a signal system, for example a coloured microparticle or colloidal gold, which will hybridise with its complementary sequence within G1′. G2 is detector agent comprising an oligonucleotide conjugated to a signal system such as a coloured microparticle or colloidal gold, which will hybridise with its complementary sequence within G2′. G3 is detector agent comprising an oligonucleotide conjugated to a signal system such as a coloured microparticle or colloidal gold, which will hybridise with its complementary sequence within G3′. G4 is detector agent comprising an oligonucleotide conjugated to a signal system such as a coloured microparticle or colloidal gold, which will hybridise with its complementary sequence within G4′.

G1′ is an oligonucleotide bridge with complementary sequence to G1 contiguous with a nucleic acid probe having a sequence that will hybridise with a second sequence within F1. G2′ is an oligonucleotide bridge with the complementary sequence to G2 contiguous with a nucleic acid probe sequence that will hybridise with a second sequence within F2. G3′ is an oligonucleotide bridge with the complementary sequence to G3 contiguous with a nucleic acid probe sequence that will hybridise with a second sequence within F3. G4′ is an oligonucleotide bridge with the complementary sequence to G4 contiguous with a nucleic acid probe sequence that will hybridise with a second sequence within F4.

FIG. 3C shows a generic representation of hybridisation events in a nucleic acid lateral flow assay. FIG. 3C shows captured analytes and reagents for a multiplexed nucleic acid detection test designed for four analytes. The analyte and reagents are described for the first line or spot (A1) on the filter only.

Analyte F1 in the sample hybridises in solution to both A′ and G1′. The oligonucleotide having sequence A140 is bound to filter A1 and captures analyte F1. F1 in turn captures detector agent G1 via the hybridisation bridge G1′.

FIG. 3D shows a strip as it would appear if nucleic acid analytes F1, F2, F3 and F4 were all detected from an amplified sample. 3D(i) shows the appearance of a positive strip if oligonucleotides A1, A2, A3 and A4 were striped onto the filter strip. 3D(ii) shows the appearance of a positive strip if oligonucleotides A1, A2, A3 and A4 were spotted onto the filter strip.

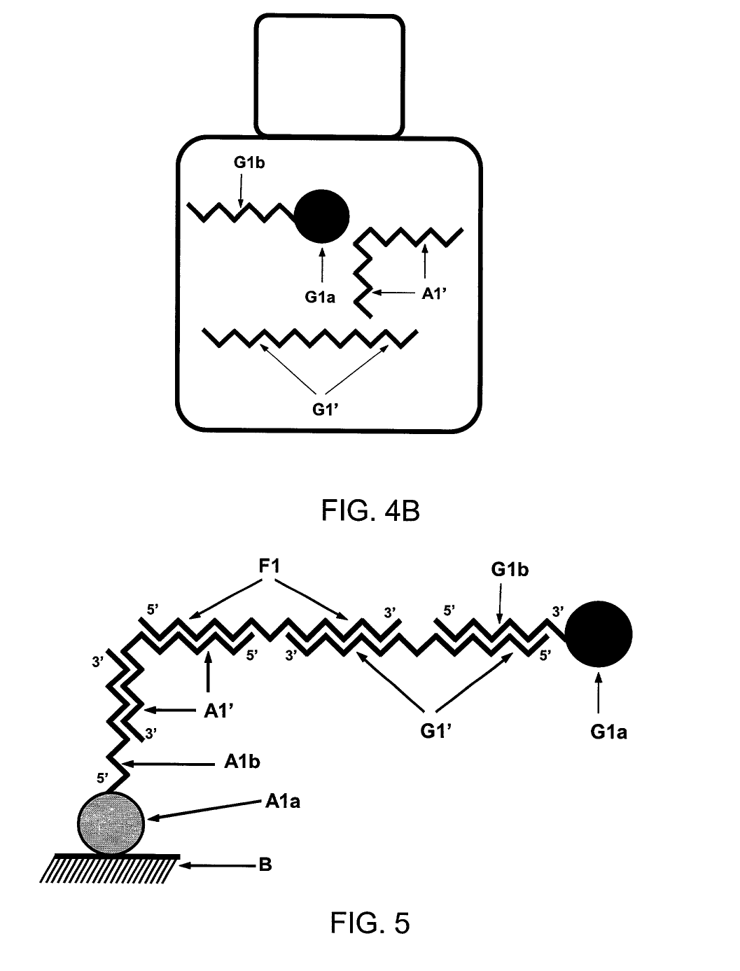

FIGS. 4A and 4B related to reagents required to perform a nucleic acid rapid lateral flow assay. FIG. 4A shows a means of attaching a receptor oligonucleotide to a filter. A1b representing an oligonucleotide sequence of the invention is conjugated to a microparticle, A1a which in turn is attached to a zone on a filter strip B.

FIG. 4B shows reagents provided for a nucleic acid detection assay. FIG. 4B shows an assay bottle with a mixture of reagents consisting of 1) a reporter agent comprising a receptor oligonucleotide G1b conjugated to a coloured microparticle, G1a and 2) bridging oligonucleotides G1′ for the capture of the reporter agent onto the analyte and A1′ for the capture of a specific analyte at a specific capture zone.

FIG. 5 shows hybridisation events in a nucleic acid detection assay using a coloured microparticle as a component of a detector or reporter agent. A1b, comprises a receptor oligonucleotide conjugated to a microparticle or other carrier particle or molecule, A1a, is attached to filter B. The nucleic acid target, F1, is captured onto the filter via hybridisation with A1′ that also hybridises to A1b. The bridging oligonucleotide G1′ hybridises to both F1 and G1b, the receptor oligonucleotide component of G1 which allows the visual reading of a positive result by the accumulation of the coloured microparticle, G1a.

The 3′ and 5′ ends of the oligonucleotides as shown may be reversed if required.

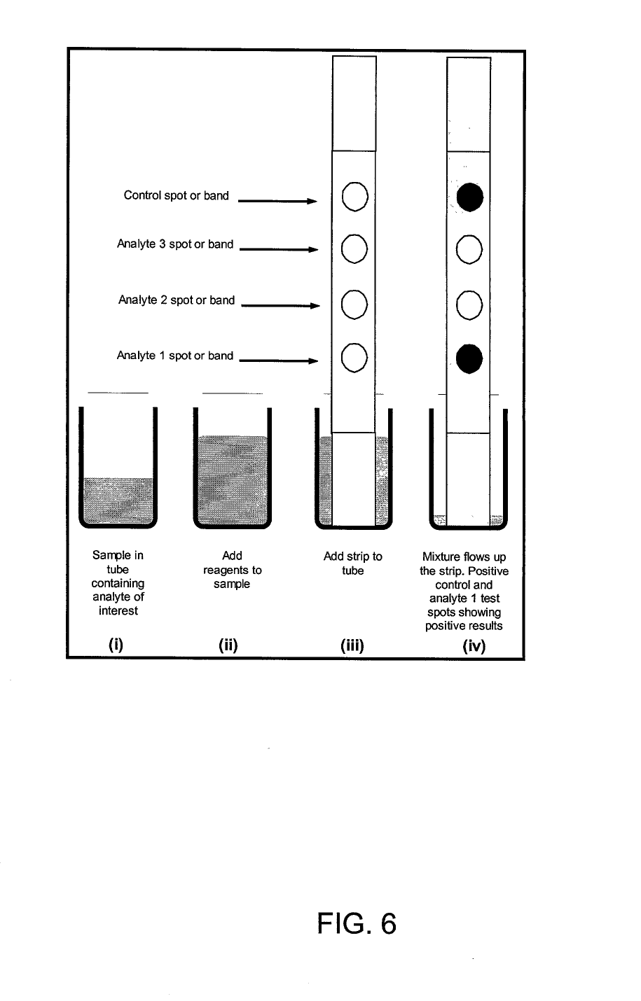

FIG. 6 represents a diagram of a test strip procedure for nucleic acid detection. The various steps involved in a nucleic acid detection assay by rapid lateral flow are highlighted as follows:

In step (i) sample prepared for nucleic acid detection thought to contain a target nucleic acid analyte(s) is placed into a tube. The nucleic acid might be derived by in vitro amplification of DNA or RNA, for example, by polymerase chain reaction or similar procedure or by biological amplification by culture procedures or by direct preparation of nucleic acids in a medical, veterinary, environmental or other sample.

In step (ii) necessary reagents for the capture and detection of a nucleic acid analyte are added to the tube and the mixture is heated to 95° C. and then slowly allowed to cool down to room temperature during which hybridisation of the bridging oligonucleotides, A1′ and G1′, and the reporter agent, G1, occurs to the target F1. Similarly, if target nucleic acid F2 is present in the mixture then reagents A2′, G2′ and reporter agent G1 hybridises to F2.

In step (iii) the test strip is added to the nucleic acid analyte(s) and now the hybridised material is allowed to wick up the strip as in a lateral flow format.

In step (iv) as the analyte is captures at a specific capture zone colour accumulates and an observable signal is generated.

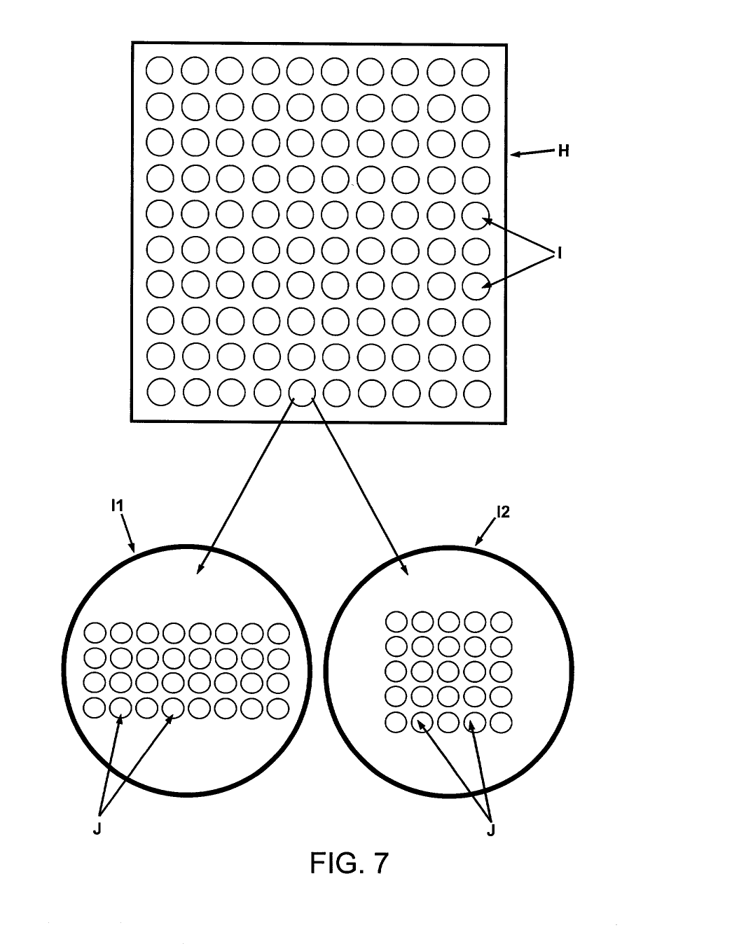

FIG. 7 shows application of nucleic acid sequences of the present invention in biochips. An example of a biochip, H, comprising a 10×10 microarray, I. Each of the 100 components of the microarray comprises 32 (I1) or 25 (I2) subarrays, J, as examples.

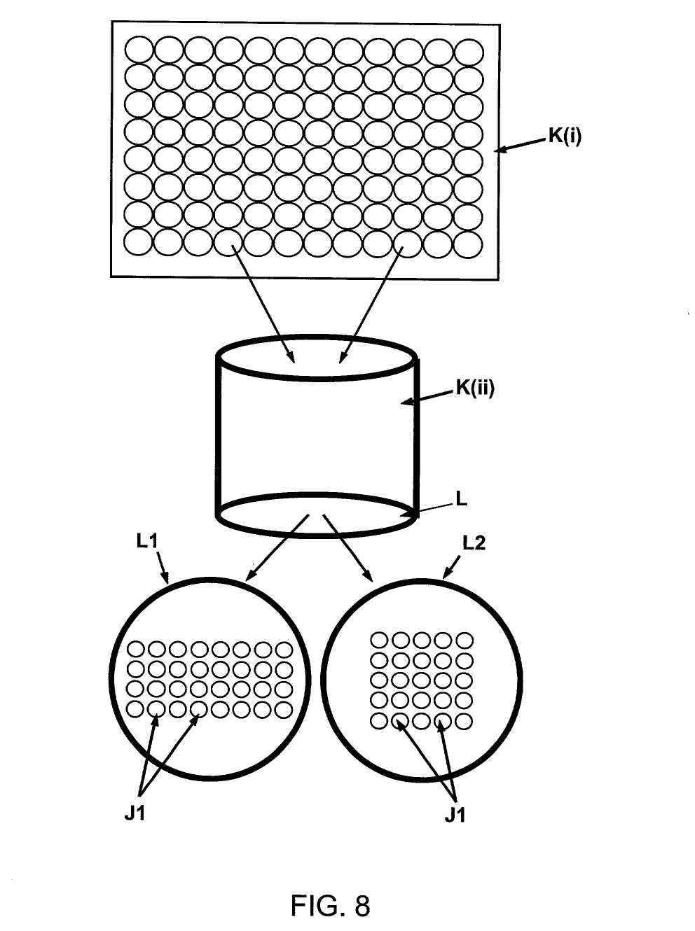

FIG. 8 shows sub-arrays may be applied to multi-well plates. Wells of a microplate K(i) with multiple wells, eg 96-well and 384-well may be used to create microarrays using receptor-binder nucleic acids. K(ii) is a well of a microplate and L is an inner surface of a base of the well. L1 provides an example of a 32-microarray with microwells identified as J1 through J32 and L2 provides an example of a 25-microarray with microwells identified as J1 through J25.

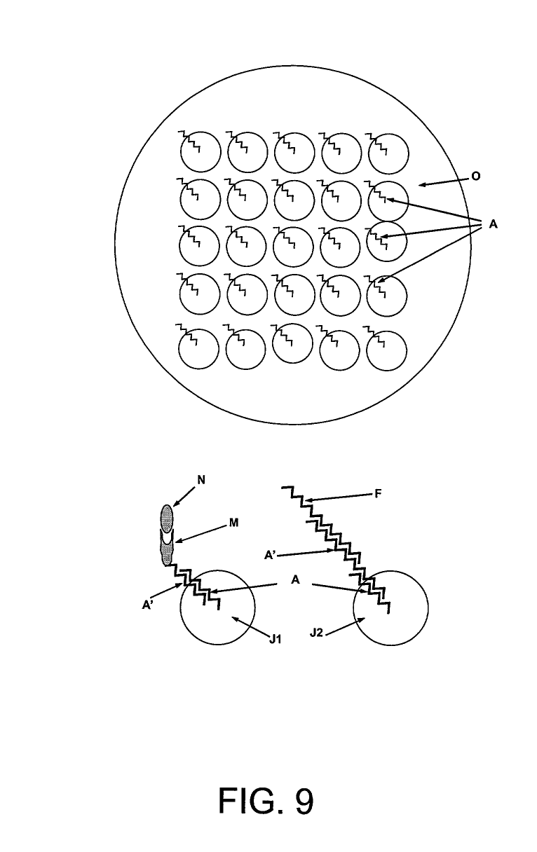

FIG. 9 shows an example of a microarray of 25 sub-wells, in either a microchip or microplate format for use in nucleic acid or protein applications. Twenty-five different receptor nucleic acids, A1 through A25, are arrayed within each microchip array or plate microwell, 0.

An example of a protein or peptide array is shown. Receptor nucleic acid A, is attached to a specific area of microwell, J1. Bridging nucleic acid, A′, is conjugated to a peptide, protein, enzyme or other biological material (M) and will hybridise to A when added to the well.

It is then possible to screen for compounds (N) that bind to or otherwise react with M and detect that reaction using methods known in the art.

An example of a nucleic acid array is also shown. The receptor nucleic acid A, is attached to a specific area of the microwell, J2. The bridging nucleic acid, A′, concatenated to an nucleic acid probe specific for a target nucleic acid sequence will hybridise to A when added to the well.

It is then possible to screen nucleic acids for target sequences that hybridise to the probe sequence of A′.

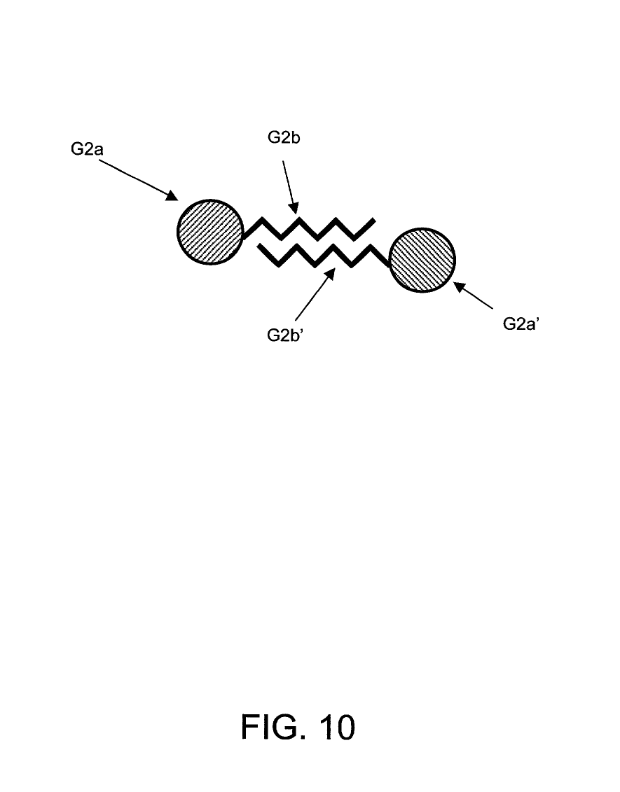

FIG. 10 shows an example of linking two species by linear isolated nucleic acids of the invention. Respective species are shown as G2a and G2a′. The species may include those described herein, for example, beads, dendrimers and proteins such as antibodies and protein subunits. G2b and G2b′ represent a complementary pair of linear isolated nucleic acids of the invention. It will be appreciated that a plurality of linear isolated nucleic acids of the invention may be attached to one or more species, thereby linking a plurality of species. The isolated nucleic acids may be attached to the species by conjugation or other suitable means.

### EXAMPLES OF APPLICATIONS

### EXAMPLE 6

Molecular and Immunoassay Diagnostics

(1) The nucleic acids having the disclosed sequences of the present invention may be used for the capture of one, two, but in particular, a multiplicity of analytes from solution on an appropriate support matrix, for example, in a microtitre plate format as described in International Patent Publication No. WO 95/24649 or in a lateral flow format as described in International Patent Publication No. WO 99/32885, herein incorporated by reference. Examples of the latter are shown in FIGS. 2 through 6

(2) Coloured latex beads, gold microparticles, fluorescent compounds, enzymes, or other reporter molecules or species may be conjugated to nucleic acid sequences of the present invention to be used as reporter agent (or signal reagent) for the detection of nucleic acid sequences, as analytes, captured on an appropriate support matrix such as microtitre plates, microchips, and lateral flow devices. The analyte nucleic acid may have the complementary sequence to the nucleic acid reporter probe incorporated either into its 5′ or 3′ end for specific hybridisation and detection. Alternatively, a chimeric nucleic acid may be used to bridge the reporter probe with the target nucleic acid sequence whereby a portion of the chimeric nucleic acid sequence hybridises specifically to the target nucleic acid. Hybridisation may occur anywhere along the nucleic acid, but preferably at a location comprising a nucleotide sequence unique to the nucleic acid, with a second location of the chimeric nucleic acid hybridising specifically to the reporter probe allowing for specific detection of the target nucleic. It will be appreciated that multiple nucleic acid analytes may be detected simultaneously at different capture zones. It will further be appreciated that a multiple of respective different reporter molecules or species may be used to provide a distinct signal for each analyte. For example, different coloured latex beads, dendrimer, or chromophores.

(3) In a different application, nucleic acids of the present invention may be used as capture probes for nucleic acid analytes. A complementary sequence to the capture probe may be incorporated into the nucleic acid analyte during the amplification process as part of the primer sequence used in the amplification. Alternatively, a chimeric nucleic acid may be used as in FIG. 2 for the hybridisation capture of the analyte nucleic acid whereby the chimeric nucleic acid recognises and hybridises with a unique sequence on the analyte nucleic acid with the other portion of the chimeric nucleic acid hybridising with the capture nucleic acid probe immobilised on the solid support. Again, using nucleic acids having the disclosed sequences of the present invention the capture of a multiplicity of nucleic acid analytes from a mixture in solution on an appropriate support matrix is enabled.

(4) The nucleic acids of the present invention may be applied as in FIGS. 2 and 3 concurrently for the capture and detection of amplified nucleic acid analytes either in a single analyte format or a multiple analyte format as for International Patent Publication No. WO 99/32885. It will be appreciated that nucleic acid analytes may be amplified by an in vitro amplification system such as polymerase chain reaction or by more conventional means such as bacterial, viral, fungal or cell culture. Where the nucleic acid analyte is captured in a well on a microtitre plate or similar device, a reporter probe may be directly attached to an enzyme if enzymatic detection is needed, similarly, where the nucleic acid target is captured in a micro-array, for example, then chromophores may be used as reporter molecule or species.

### EXAMPLE 7

Array Technology

(1) The nucleic acids of the present invention may be attached to an appropriate support matrix such as glass, plastic or other polymeric material, gel elements fixed on a solid support (Mirzabekov, A., et al., 1997, Analytical Biochemistry, 250, 203; Dubiley, S., et al., 1996, Proc. Natl. Acad. Sci. USA, 93, 4913) or coated silicon (Polisky, B., et al., 2001, Nature Biotechnology, 19, 62) herein incorporated by reference for the purpose of developing a generic nucleic acid micro-array (Fodor, S. P. A., 1997, Science, 277, 393) herein incorporated by reference. The micro-array may be for example, on a microchip as described by Fodor, (Fodor, S. P. A., et al., 1995, BioTechniques 19, 442, herein incorporated by reference) or in the form of a 96-well micro-array plate as described by Eggers, (Eggers, M., et al., 1999, BioTechniques, 27, 778) or as described by Dubiley (Dubiley, S., et al., 1996, Proc. Natl. Acad. Sci. USA., 93, 4913) which are all incorporated herein by reference. It will be appreciated that 96, 384-, or 1536-, etc. well plates may be used.

(2) Nucleic acids of the present invention may be attached to coloured or fluorescent dye coded beads or another suitable tagging system to generate a generic “array” whereby each unique oligonucleotide sequence of the present invention is identified by the coloured bead or label it is attached to. An assay performed using each specific oligonucelotide of the present invention in an analogues manner to microarrays where a location of an oligonucelotide in a 2-dimensional array is usually used to identify that specific oligonucelotide. Examples of such colour coded beads may be found in International Patent number WO 99/24458 and U.S. Pat. No. 6,268,222, both incorporated herein by reference. Beads are commercially available from companies including Luminex Corporation, Austin, Tex., USA. Up to 100 different colour coded beads incorporating 2 different dyes in different proportions are available. For a set of 32 pairs of oligonucelotides of the present invention an “array” of 3200 is possible. The incorporation of a third dye in each bead will allow for an “array” of 32000. Assays using these beads may then be interrogated, in principle, as described in U.S. Pat. No. 6,139,800, incorporated herein by reference. It will be appreciated that oligonucelotides of the present invention may be attached to a support matrix in a 2-dimensional array or on beads through a hairpin system as described in U.S. Pat. No. 5,770,365, incorporated herein by reference, where the linear portion of the hairpin capture probe is comprised of oligonucleotides of the present invention which hallows more efficient hybridisation capture. Nucleic acids having complementary sequences to nucleic acids immobilised in a 2-dimensional array or label coded beads may be attached to a plurality of ligands for use in diagnostic assays or high throughput screening. Examples of such ligands, include but are not limited to antibodies and fragments thereof, antigens, peptide, proteins, enzymes, amplified nucleic acids, RNA, DNA, PNA, synthetic DNA analogues, haptens, pharmaceutical drugs, drugs of abuse, and neutraceutical compounds.

(3) The nucleic acids of the present invention may be conjugated to a variety of different surface groups to produce different surface chemistries for genomics or proteomics applications. Such groups include but are not restricted to hydrophobic groups, hydrophilic groups, groups with cationic or anionic charges, etc. This may allow capture of different proteins from a mixture at specific microspots in an array for interrogation, they may equally be captured on label-coded beads as described above. Other such applications may include the capture of expressed proteins such as enzymes, antibodies, antigens, growth factors, hormones, etc. whereby these proteins are produced attached to the coding RNA or DNA sequence (Roberts, W. A., 1999, Curr. Opin. Chem. Biol., 3, 268-273 and references therein which are incorporated herein by reference). A part of this sequence can be manipulated to comprise a sequence from any of the disclosed nucleotide sequences of the present invention allowing the protein to be captured by hybridising to its complementary sequence located at a specific microspot or label coded bead. Since each capture sequence can be related to a particular protein, then each sequence can serve as a tag for that particular protein and be identified at a specific spot in the capture zone or bead.

### EXAMPLE 8

High Throughput Screening

The nucleic acids of the present invention may be applied in antisense technology for discovering nucleic acids having nucleotide sequences as lead antisense pharmaceutical compounds. For example, nucleic acids having overlapping antisense nucleotide sequences for a particular RNA target may be immobilised on an appropriate support matrix in a microarray format using the nucleic acids of the present invention. Each candidate nucleotide sequence to be tested may comprise a tail nucleic acid having a sequence complementary to a nucleic acid immobilised on the support matrix or label coded bead, thus allowing for the capture and immobilisation of each nucleic acid at a specific location in the array. The nucleic acids having antisense nucleotide sequences are then exposed to the target RNA and the efficiency of hybridisation of the candidate nucleic acid having an antisense sequence to the target RNA is measured whereby nucleic acids that do not bind to the target RNA are detected by the introduction of a reporter probe which binds to unreacted antisense sequences.

### EXAMPLE 9

Cross-Linking and Signal Amplification

Nucleic acids of the invention may be used to link two or more species, for example proteins or beads as shown in FIG. 10.

Reference may be made to International Patent Publication No. WO 98/04740, herein incorporated by reference, which provides a method for detecting nucleic acid using nucleic acids attached to nanoparticles for visual detection of a nucleic acid target. Nucleic acids of the present invention may be attached to the nanoparticles and used in a similar way to bring about nanoparticle accumulation around the target nucleic acid.

Further reference may be made to International Patent Publication No. 00/28088, herein incorporated by reference, which discloses use of nanocrystals with nucleic acids having specific sequences attached thereto used for detection of a target molecule. The nucleic acids of the present invention may also be used in this or similar applications.

Yet in another invention as described in U.S. Pat. No. 5,965,133, which is incorporated herein by reference, a target molecule can be identified using constructs comprising multimeric forms of nucleic acid by formation of nucleic acid polymeric aggregates. Nucleic acids of the present invention may also be used in this or similar applications.

It will be appreciated that the present invention provides a nucleic acid which lacks internal secondary structure and thus retains a linear structure over a range of hybridisation conditions, including at least low stringency conditions, regardless of nucleic acid length. Also, cross-hybridisation of nucleic acids of the invention is minimal or absent regardless of nucleic acid length even at low stringency conditions.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated by reference in its entirety.

1. An isolated nucleic acid comprising a repeated nucleotide sequence of at least two different nucleotide bases having a minimal repeating nucleotide sequence of three or more nucleotide bases, whereby said nucleic acid is characterised by:
i) lacking an internal secondary structure;
ii) capable of hybridising with a complementary nucleic acid; and
iii) does not hybridise with a non-complementary nucleic acid; under at least low stringency conditions.

2. The isolated nucleic acid of claim 1 wherein said at least two different nucleotide bases are grouped so that the repeating nucleotide sequence comprises a series of adjacent A or T nucleotide bases followed by a series of adjacent G or C nucleotide bases.

3. The isolated nucleic acid of claim 1 wherein said at least two different nucleotide bases are grouped so that the repeating nucleotide sequence comprises a series of adjacent G or C nucleotide bases followed by a series of adjacent A or T nucleotide bases.

4. The isolated nucleic acid of claim 2 wherein said nucleic acid is selected from the group consisting of:
5′ TATGCGGCG TATGCGGCG 3′
5′ TTAAATGGC TTAAATGGC 3′
5′ TATTATCCCCCG TATTATCCCCCG 3′

5. The isolated nucleic acid of claim 1 wherein said nucleic acid comprises a repeating nucleotide sequence of three to twelve nucleotide bases.

6. The isolated nucleic acid of claim 5 wherein said nucleic acid comprises a repeating nucleotide sequence of six nucleotide bases.

7. An isolated nucleic acid consisting essentially of a nucleotide sequence as defined by a formula selected from the group consisting of: I, II, Ia, IIa, Ib and IIb, wherein the respective formulas are as follows:
5′ Y₁[(X₁X₂X₃X₄X₅X₆)]ₙY₂3′ (I)
3′ Y₁[(X₁X₂X₃X₄X₅X₆)]ₙY₂5′ (II)
wherein:
(A) when X₁, X₂ and X₃ are either A or T
then X₄, X₅ and X₆ are either G or C; or
(B) when X₁, X₂ and X₃ are either G or C
then X₄, X₅ and X₆ are either A or T;
n≧2
Y₁ is selected from the group consisting of:
X₂ X₃ X₄ X₅ X₆
X₃ X₄ X₅ X₆
X₄ X₅ X₆
X₅ X₆
X₆ and zero
Y₂ is selected from the group consisting of:
X₁ X₂ X₃ X₄ X₅
X₁ X₂X₃X₄
X₁X₂X₃
X₁X₂
X₁ and zero
5′ Y₁[(X₁X₂X₃X₄X₅)]ₙY₂3′ (Ia)
3′ Y₁[(X₁X₂X₃X₄X₅)]ₙY₂5′ (IIa)
wherein:
(A) when X₁, X₂ and X₃ are either A or T
then X₄ and X₅ are either G or C;
(B) when X₁, X₂ and X₃ are either G or C
then X₄ and X₅ are either A or T;
(C) when X₁ and X₂ are either A or T
then X₃, X₄ and X₅ are either G or C; or
(D) when X₁ and X₂ are either G or C
then X₃, X₄ and X₅ are either A or T;
n≧2
Y₁ is selected from the group consisting of:
X₂ X₃ X₄ X₅
X₃ X₄ X₅
X₄ X₅
X₅ and zero
Y₂ is selected from the group consisting of:
X₁ X₂ X₃ X₄
X₁ X₂X₃
X₁X₂
X₁
X₁ and zero
5′ Y₁[(X₁X₂X₃X₄)]ₙY₂3′ (Ib)
3′ Y₁[(X₁X₂X₃X₄)]ₙY₂5′ (IIb)
wherein:
(A) when X₁ and X₂ are either A or T
then X₃ and X₄ are either G or C;
(B) when X₁ and X₂ are either G or C
then X₃ and X₄ are either A or T;
n≧2
Y₁ is selected from the group consisting of:
X₂ X₃ X₄
X₃ X₄
X₄ and zero
Y₂ is selected from the group consisting of:
X₁ X₂ X₃
X₁ X₂
X₁ and zero

8. The isolated nucleic acid of claim 7 wherein n=2-20.

9. The isolated nucleic acid of claim 8 wherein n=2-6.

10. The isolated nucleic acid of claim 7 selected from the group consisting of:
5′ GC(TAACGC)₄T 3′;
5′ TT(CCCTTT)₄CCCTT 3′;
5′ (TATGGC)₄TAT 3′;
5′ AACCG(TAACCG)₄T 3′;
5′ G(TTACCG)₄TT 3′;
5′ (ATTGGG)₆ 3′;
5′ GC(TACGC)₄T 3′;
5′ TT(CCCTT)₄CCCT 3′;
5′ (GCTA)₄GCT 3′;
5′ ACC(TACC)₆T 3′;
5′ CCCTAA CCCTAACCCTAACCCTAACCCTAACCCTAA 3′;
5′ CGGAATCGGAATCGGAATCGGA TCGGAATCGGAAT 3′; and
5′ ATTCCGATTCCGATTCCG 3′

11. The isolated nucleic acid of claim 7 selected from the group consisting of:

| | | |
|---|---|---|
| 5′ (AAAGGG)ₙ 3′ | 5′ (AAAGGC)ₙ 3′ | 5′ (AAAGCG)ₙ 3′ |
| 5′(AAAGCC)ₙ 3′ | | |
| 5′ (AAACCC)ₙ 3′ | 5′ (AAACCG)ₙ 3′ | 5′ (AAACGC)ₙ 3′ |
| 5′(AAACGG)ₙ 3′ | | |
| 5′ (TTTGGG)ₙ 3′ | 5′ (TTTGGC)ₙ 3′ | 5′ (TTTGCG)ₙ 3′ |
| 5′(TTTGCC)ₙ 3′ | | |
| 5′ (TTTCCC)ₙ 3′ | 5′ (TTTCCG)ₙ 3′ | 5′ (TTTCGC)ₙ 3′ |
| 5′(TTTCGG)ₙ 3′ | | |
| 5′ (AATGGG)ₙ 3′ | 5′ (AATGGC)ₙ 3′ | 5′ (AATGCG)ₙ 3′ |
| 5′(AATGCC)ₙ 3′ | | |
| 5′ (AATCCC)ₙ 3′ | 5′ (AATCCG)ₙ 3′ | 5′ (AATCGC)ₙ 3′ |
| 5′(AATCGG)ₙ 3′ | | |
| 5′ (TTAGGG)ₙ 3′ | 5′ (TTAGGC)ₙ 3′ | 5′ (TTAGCG)ₙ 3′ |
| 5′(TTAGCC)ₙ 3′ | | |
| 5′ (TTACCC)ₙ 3′ | 5′ (TTACCG)ₙ 3′ | 5′ (TTACGC)ₙ 3′ |
| 5′(TTACGG)ₙ 3′ | | |
| 5′ (TAAGGG)ₙ 3′ | 5′ (TAAGGC)ₙ 3′ | 5′ (TAAGCG)ₙ 3′ |
| 5′(TAAGCC)ₙ 3′ | | |
| 5′ (TAACCC)ₙ 3′ | 5′ (TAACCG)ₙ 3′ | 5′ (TAACGC)ₙ 3′ |
| 5′TAACGG)ₙ 3′ | | |
| 5′ (ATTGGG)ₙ 3′ | 5′ (ATTGGC)ₙ 3′ | 5′ (ATTGCG)ₙ 3′ |
| 5′(ATTGCC)ₙ 3′ | | |
| 5′ (ATTCCC)ₙ 3′ | 5′ (ATTCCG)ₙ 3′ | 5′ (ATTCGC)ₙ 3′ |
| 5′(ATTCGG)ₙ 3′ | | |
| 5′ (ATAGGG)ₙ 3′ | 5′ (ATAGGC)ₙ 3′ | 5′ (ATAGCG)ₙ 3′ |
| 5′(ATAGCC)ₙ 3′ | | |
| 5′ (ATACCC)ₙ 3′ | 5′ (ATACCG)ₙ 3′ | 5′ (ATACGC)ₙ 3′ |
| 5′(ATACGG)ₙ 3′ | | |
| 5′ (TATGGG)ₙ 3′ | 5′ (TATGGC)ₙ 3′ | 5′ (TATGCG)ₙ 3′ |
| 5′(TATGCC)ₙ 3′ | | |
| 5′ (TATCCC)ₙ 3′ | 5′ (TATCCG)ₙ 3′ | 5′ (TATCGC)ₙ 3′ |
| 5′(TATCGG)ₙ 3′ | | |

12. The isolated nucleic acid of claim 1 or claim 7 wherein said respective A, T, C or G nucleotide base is a derivative thereof.

13. The isolated nucleic acid of claim 1 or claim 7 wherein two or more isolated nucleic acids comprising a same number of nucleotide bases are characterised by a Tₘ substantially identical or similar to each other.

14. The isolated nucleic acid of claim 1 or claim 7 further comprising one or more linkers attached to or contiguous with any respective one or more nucleotide bases thereof.

15. The isolated nucleic acid of claim 14 wherein the linker is selected from the group consisting of: a substituted or unsubstituted alkyl group having one or more carbons, wherein the alkyl groups may be linear or branched; a substituted or unsubstituted aryl or aryl alkyl group; proteins and peptides, including linear and branched peptides; peptides comprising a lysine residue; nucleic acids, including oligonucleotides and primers; dendrimers or dendrimeric like molecules; polymers; oligomers comprising a plurality of units; and other linear polymeric materials.

16. The isolated nucleic acid of claim 14 wherein the isolated nucleic acid comprises a nucleotide sequence that is contiguous with a nucleotide sequence of the linker.

17. The isolated nucleic acid of claim 14 wherein one or more species is respectively attached to or is contiguous with the one or more linkers.

18. The isolated nucleic acid of claim 1 or claim 7 further comprising one or more species respectively attached to any one or more nucleotide bases thereof.

19. The isolated nucleic acid of claim 18 wherein said species is an expressed protein.

20. A nucleic acid structure comprising a structure defined by a formula selected from the group consisting of: 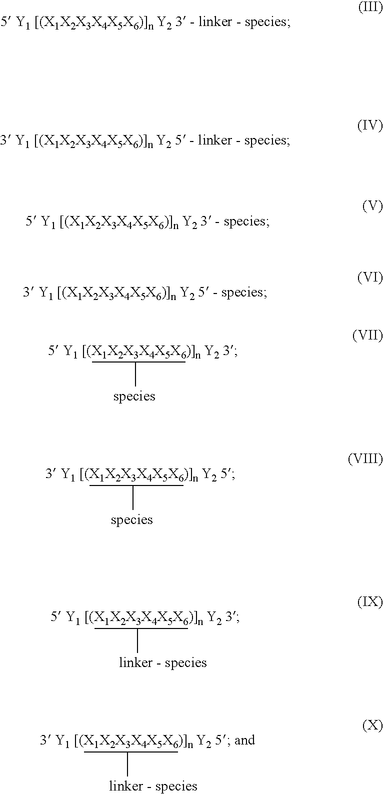
where in formulae (VII) to (X) the species may be attached to any one or more nucleotide base thereof.

21. The isolated nucleic acid according to claim 1 or claim 7 when used as a linker for cross linking two or more species in solution, at a mixed interphase, at a solid-solid phase interface, or combination thereof, by hybridising a complementary binding pair of said isolated nucleic acids wherein each respective complementary isolated nucleic acid is attached to at least one of said two or more species.

22. The isolated nucleic acid of claim 20 wherein said species is selected from the group consisting of: antibodies, antibody fragments including a light chain fragment, antibody mimetics, antigens or parts thereof, antigen mimetics, enzymes, proteins, peptides, organic molecules, haptens, pharmaceutical compounds, dendrimers and dendrimeric-like molecules, coloured dendrimers, beads, coloured beads, latex beads, microparticles or other coloured polymeric or branched materials, gold microparticles, reporter molecules, fluorochromes (or fluorescent compounds), dyes, metal chelates, radioactive isotopes, nucleic acids including oligonucleotides and nucleic acid amplification products including PCR products, RNA, DNA, PNA and synthetic oligonucleotides.

23. The isolated nucleic acid of claim 22 wherein one or more isolated nucleic acid(s) comprising a nucleotide sequence 5′ ATTCCGATTCCGATTCCG 3′ are attached to one or more coloured latex bead(s).

24. The isolated nucleic acid of claim 19 wherein said one or more species is respectively attached to one or more nucleotide base(s) internal of terminal ends of said isolated nucleic acid.

25. The isolated nucleic acid of claim 24 comprising multiple reporter molecules.

26. The isolated nucleic acid of claim 25 when used as a signal reagent for detecting one or more target(s).

27. The isolated nucleic acid according to claim 1 or claim 7 wherein either a 3′ or 5′ end of said isolated nucleic acid is immobilised to a support and said isolated nucleic acid is capable of hybridising with one or more target(s).

28. The isolated nucleic acid of claim 27 wherein said isolated nucleic acid comprises a nucleotide sequence 5′CCCTAA CCCTAACCCTAACCCTAACCCTAACCCTAA 3′ or 5′ CGGAATCGGAATCGGAATCGGAATCGGAATCGGAAT 3′.

29. The isolated nucleic acid of claim 27 wherein said support is selected from the group consisting of: a membrane, a microarray chip, and surface of a dish, well, insoluble support matrix, microtiter plate or microparticle.

30. The isolated nucleic acid of claim 29 wherein said isolated nucleic acids are immobilised to a support used with a lateral flow assay.

31. An array comprising:
A) a support; and
B) one or more isolated nucleic acids of claim 1 or claim 7 immobilised to the support.

32. The array of claim 31 wherein the support is selected from the group consisting of: glass, plastic, polymeric material and a gel.

33. The array of claim 31 wherein the array is a microarray or a multi-well plate.

34. The array of claim 31 wherein the support is a labelled microparticle.

35. The array of claim 34 wherein the labelled microparticle is a labelled bead or labelled dendrimer.

36. The array of claim 34 wherein the labelled microparticle is a coloured microparticle.

37. The array of claim 34 wherein the one or more isolated nucleic acid(s) comprise a same nucleotide sequence.

38. The array of claim 34 wherein the array is characterised as a generic array in solution.

39. A method for capturing one or more target(s) which includes the step of immobilising one or more isolated nucleic acids of claim 1 or claim 7 to a support, wherein each immobilised isolated nucleic acid is capable of hybridising with a complementary nucleic acid.

40. A method of linking two or more species, the method including the step of respectively attaching an isolated nucleic acid of claim 1 or claim 7 to said respective two or more species, wherein said isolated nucleic acids are capable of forming a complementary binding pair.

41. A method of making a signal reagent, said method including the step of attaching one or more reporter molecules to an isolated nucleic acid of claim 1 or claim 7.
